(19) United States
(12) Patent Application Publication
(10) Pub. No.: US 2026/0097220 A1
(43) Pub. Date: Apr. 9, 2026

(54) MULTI-SHOCK ACCESSORY DEVICE

(71) Applicant: Stryker Corporation, Portage, MI (US)

(72) Inventors: David J. Linville, Redmond, WA (US); Ryan William Apperson, Bothell, WA (US); Fred W. Chapman, Newcastle, WA (US); Rockland W. Nordess, Kirkland, WA (US); Reza Sharif, Lake Forest, WA (US); Tyson G. Taylor, Bothell, WA (US); Robert G. Walker, Seattle, WA (US); Rose Tingwei Yin, Bellevue, WA (US)

(73) Assignee: Stryker Corporation, Portage, MI (US)

(21) Appl. No.: 19/352,291

(22) Filed: Oct. 7, 2025

Related U.S. Application Data
(60) Provisional application No. 63/705,472, filed on Oct. 9, 2024.

(51) Int. Cl. A61N 1/39; A61N 1/02; A61N 1/04

(52) U.S. Cl. CPC A61N 1/39044; A61N 1/025; A61N 1/046; A61N 1/3925; A61N 1/3968; A61N 1/3987

(57) ABSTRACT

An example method includes receiving, by a multi-shock accessory device, a multi-shock instruction from a defibrillator, the multi-shock instruction indicating a time interval between a primary electrical shock and a secondary electrical shock. In response to receiving the multi-shock instruction, the example method includes causing, by the multi-shock accessory device, a treatment circuit of the multi-shock accessory device to output a secondary electrical shock to electrodes disposed on skin of a subject; or causing, by the multi-shock accessory device, a capacitor of the multi-shock accessory device to discharge the secondary electrical shock to the electrodes.

### CROSS-REFERENCE TO RELATED APPLICATION

This application claims the priority of U.S. Provisional App. No. 63/705,472, which was filed on Oct. 9, 2024 and is incorporated by reference herein in its entirety.

### BACKGROUND

Particular heart arrhythmias, such as ventricular fibrillation (VF) and pulseless ventricular tachycardia (VT), are deadly if untreated. An individual with one of these arrhythmias can be treated by administering an electrical shock to the individual's heart. This treatment is known as defibrillation. The arrhythmias that are treatable by defibrillation are known as shockable arrhythmias.

Unfortunately, some instances of shockable arrhythmias are resistant to conventional defibrillation therapies. For instance, an individual is determined to have refractory VF if they have VF that continues through the administration of an electrical shock. Recently, vector change and double sequential defibrillation (DSD) (also referred to as “double sequential external defibrillation” or “DSED”) have been proposed as an alternative to single-shock defibrillation. A DSD therapy, for instance, can be administered by discharging two electrical shocks to an individual's heart, rather than one. Researchers have suggested that DSD administration can increase survivability of refractory VF. Cheskes et al., 387 N. Engl. J. Med. 1947 (2022).

### BRIEF DESCRIPTION OF THE DRAWINGS

FIG. 1 illustrates an environment in which a subject is treated with a multi-shock therapy by two separate devices.

FIG. 2 illustrates example signaling for coordinating a multi-shock therapy.

FIG. 3 illustrates an example of a primary defibrillator configured to physically connect with a multi-shock accessory.

FIG. 4 illustrates an example of an electrode pad that is coupled with a multi-shock accessory.

FIG. 5A illustrates an example process for administering a multi-shock therapy based on an instruction from a defibrillator.

FIG. 5B illustrates an example process for coordinating a multi-shock therapy with defibrillator.

FIG. 6 illustrates an example of an external defibrillator configured to perform various functions described herein.

FIG. 7 illustrates a chest compression device configured to perform various functions described herein.

FIGS. 8A and 8B illustrate examples of environments and timing related to administering a multi-shock therapy. FIG. 8A shows an environment configured to administer the multi-shock therapy. FIG. 8B shows a timing relationship of multiple shocks administered in the multi-shock therapy.

### DETAILED DESCRIPTION

Various implementations described herein relate to techniques for improving the administration and coordination of multi-shock therapies (e.g., DSD therapies) using multiple devices. For instance, a user could administer a DSD therapy to an individual by manually activating two separate defibrillators connected to the individual. However, manually activating two separate defibrillators has some drawbacks. Namely, certain timing relationships between electrical shocks may reduce the efficacy of the DSD therapy, or even cause the DSD therapy to have lower efficacy than therapy from a single defibrillator. Moreover, it is possible that an electrical shock output by one defibrillator can harm sensitive circuitry of the other defibrillator during administration of the DSD therapy. Furthermore, manually transporting both defibrillators to the scene of the individual could be challenging, particularly for individual rescuers. Additionally, if both defibrillators are configured to monitor the individual, it could be difficult for rescuers to parse alarms and other signals output by both defibrillators, simultaneously, at a rescue scene.

Implementations of the present disclosure address these and other problems by utilizing a multi-shock accessory. In some examples, the multi-shock accessory has a defibrillation capability but limited functionality compared to a standalone defibrillator (e.g., a monitor-defibrillator or automated external defibrillator (AED)). The multi-shock accessory, for instance, is configured to administer one of the electrical shocks in a multi-shock therapy without being configured to output alarms, monitor the individual being treated, or to otherwise distract a rescuer from focusing on information output by a primary defibrillator at the scene. In some cases, the multi-shock accessory is a standalone defibrillator with one or more functions that have been deactivated based on an instruction by the primary defibrillator. The multi-shock accessory, in various cases, is configured to be physically coupled with the primary defibrillator, to wirelessly pair with the primary defibrillator, or the like. Based on a signal from the primary defibrillator, the multi-shock accessory is configured to output an electrical shock to the individual, for instance.

Various implementations will now be described with reference to the accompanying figures.

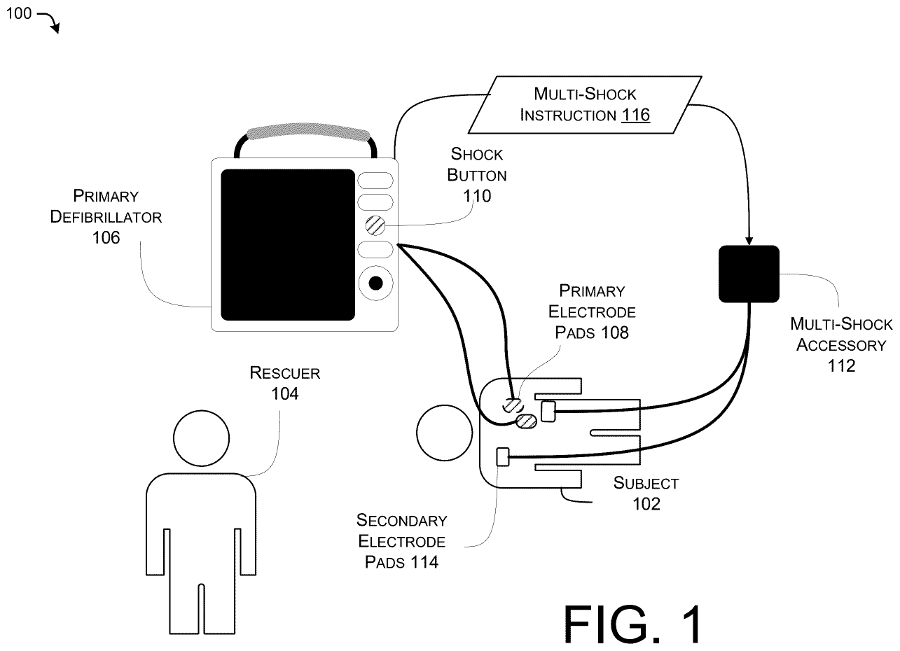

FIG. 1 illustrates an environment 100 in which a subject 102 is treated with a multi-shock therapy by two separate devices. In various cases, the environment 100 is in an out-of-hospital environment. For example, the subject 102 may have experienced a medical emergency in a non-hospital, public space, such as a library, airport terminal, school, or office building. In some cases, the subject 102 has collapsed or otherwise lost consciousness within the environment 100. In some examples, the subject 102 has experienced one or more other types of symptoms associated with a serious health condition. As a result, a bystander may have contacted emergency services personnel in order to assist the subject 102 and to transport the subject 102 to a clinical environment, if necessary.

In various cases, a rescuer 104 operates a primary defibrillator 106 in order to monitor and potentially treat the subject 102. In various cases, the primary defibrillator 106 is a monitor-defibrillator, AED, or other defibrillator device. In some instances, the primary defibrillator 106 is designed for operation by users without specialized medical knowledge. In some examples, the rescuer 104 has specific medical expertise. For instance, the rescuer 104 is an emergency medical services (EMS) professional, a physician, a nurse, or some other individual with specific medical training.

In various cases, the primary defibrillator 106 instructs the rescuer 104 to apply primary electrode pads 108 to the skin of the subject 102. For instance, the primary electrode pads 108 may be adhered to skin on the chest of the subject 102. In various cases, under direction of the primary defibrillator 106, the rescuer 104 applies the primary electrode pads 108 to the subject 102 along a first shock vector.

The primary defibrillator 106 includes one or more measurement circuits configured to detect one or more physiological parameters of the subject 102. The measurement circuit(s), for instance, are configured to be connected to sensors that are applied to the body of the subject 102. The sensor(s), for instance, are configured to generate electrical signals that are indicative of the physiological parameter(s) of the subject 102. The measurement circuit(s) may infer the physiological parameter(s) based on the electrical signals generated by the sensor(s).

In particular cases, the primary electrode pads 108 include electrodes that are configured to detect an electrical signal output by the heart of the subject 102 over time. A measurement circuit in the primary defibrillator 106, for instance, is configured to detect an electrocardiogram (ECG) of the subject 102 based on the electrical signal detected by the electrodes.

In various implementations, the primary defibrillator 106 is configured to identify a condition of the subject 102 based, at least in part, on the physiological parameter(s). For instance, the primary defibrillator 106 may identify that the subject 102 is experiencing a shockable arrhythmia by analyzing the ECG. Optionally, the primary defibrillator 106 identifies that the subject 102 is experiencing the shockable rhythm by also analyzing an additional physiological parameter, such as blood pressure, blood oxygenation, or the like. Shockable arrhythmias include any arrhythmia that is treatable by administration of an electrotherapy. For example, some shockable arrhythmias, such as ventricular fibrillation (VF) or pulseless ventricular tachycardia (VT), are treatable by the administration of one or more electrical shocks (e.g., in a range of 50 Joules (J) to 360 J). Bradycardia (e.g., a heart rate, as noted by QRS complexes in the ECG, that is below a threshold) is an example of an arrhythmia that is treatable by the administration of pacing pulses. Atrial fibrillation (AF) is an example of a shockable arrhythmia that is treatable by the administration of one or more electrical shocks that are synchronized with one or more QRS complexes or R waves identified in the ECG. This treatment for AF is also referred to as synchronized cardioversion.

For example, the primary defibrillator 106 may infer that the subject 102 is experiencing pulseless VT by determining that the ECG of the subject 102 is indicative of tachycardia and a lower-than-a-threshold blood pressure of the subject 102 indicates that the heart of the subject 102 is not spontaneously pumping blood through the body of the subject 102.

In some examples, the primary defibrillator 106 is configured to output, to the rescuer 104, a recommendation to administer a treatment to the subject 102. For example, the primary defibrillator 106 may output audible instructions, visual instructions, or a combination thereof, to administer the treatment. In some examples, the primary defibrillator 106 instructs the rescuer 104 to administer chest compressions to the subject 102. According to some cases, the primary defibrillator 106 coaches the rescuer 104 on administering effective chest compressions. For example, the primary defibrillator 106 may output an indication of a rate, depth, position, or other treatment parameter associated with optimizing the chest compressions for circulating blood through the body of the subject 102. In some implementations, the chest compressions are administered by a mechanical chest compression device.

In various cases, the primary defibrillator 106 is configured to output a recommendation to administer an electrical shock to the subject 102. For example, the primary defibrillator 106 is configured to output audible instructions, visual instructions, or a combination thereof, to press a shock button 110 on the primary defibrillator 106 that will cause a first treatment circuit in the primary defibrillator 106 to output an electrical shock to the primary electrode pads 108. The electrical shock, for example, is a defibrillating electrical shock. For example, the primary defibrillator 106 outputs the instruction in response to detecting the shockable arrhythmia. Once the rescuer 104 presses the shock button 110, the primary defibrillator 106 outputs the electrical shock to the heart of the subject along the first shock vector via the primary electrode pads 108.

In various implementations, the medical condition of the subject 102 persists and/or recurs after administration of the treatment. In particular cases, the subject 102 has VF that is resistant to attempted defibrillation by the primary defibrillator 106. For example, the VF of the subject 102 may temporarily resolve in response to the primary defibrillator 106 delivering the electrical shock to the primary electrode pads 108. However, the VF of the subject 102 may recur after the electrical shock is delivered (e.g., recurrent VF). In some cases, the VF of the subject does not even temporarily abate in response to administration of the electrical shock (e.g., refractory VF). In various cases, the subject 102 has a treatment-resistant shockable arrhythmia.

The rescuer 104 and/or the primary defibrillator 106, in various cases, determine that the serious medical condition of the subject 102 can be addressed by a multi-shock therapy, such as a double-sequential defibrillation (DSD) therapy. As used herein, the term “multi-shock therapy,” and its equivalents, refers to the administration of at least two electrical shocks to a subject in order to treat a shockable arrhythmia of the subject. In various cases, at least one of the electrical shocks has an energy level in a range of 50 J to 360 J. In some cases, each of the electrical shocks has an energy level in the range of 200 J to 360 J. In some cases, the electrical shocks are sequentially applied, such that one electrical shock ends before the other begins. In various implementations, the electrical shocks of a multi-shock therapy are temporally overlapping. For example, a leading edge of a second electrical shock occurs after the leading edge of a first electrical shock and before the trailing edge of the first electrical shock. In an example multi-shock therapy, there is at least one time interval in which the subject receives two defibrillating shocks simultaneously. In various cases, each of the electrical shocks is multiphasic (e.g., biphasic). Although FIG. 1 is primarily described with reference to a multi-shock therapy, other multi-shock therapies can also be administered in the environment 100. For example, in various cases, electrical shocks can be administered sequentially as part of a pacing therapy or as part of a synchronized cardioversion therapy.

Various characteristics of the subject 102 indicate that the multi-shock therapy is warranted. In some cases, the multi-shock therapy is associated with greater risks to the subject 102 than a conventional electrical shock. For example, the multi-shock therapy may be associated with a higher risk of burns than a single-shock therapy. Accordingly, the multi-shock therapy may be indicated in only a subset of types of patient conditions associated with shockable arrhythmias.

In particular cases, the rescuer 104 and/or the primary defibrillator 106 determines that the multi-shock therapy is warranted in response to determining that the subject 102 has a treatment-resistant shockable arrhythmia, such as refractory VF. For example, the rescuer 104 and/or the primary defibrillator 106 may determine that the subject 102 has a persistent shockable arrhythmia in spite of being administered with one or more previous electrical shocks from the primary defibrillator 106. In some cases, the primary defibrillator 106 receives a communication signal from an external device (e.g., an AED that has already monitored and attempted to treat the subject 102) indicating the treatment-resistant shockable arrhythmia. According to various implementations, the rescuer 104 and/or the primary defibrillator 106 may analyze one or more physiological parameters of the subject 102 in order to determine that the multi-shock therapy is warranted. According to various cases, the rescuer 104 and/or the primary defibrillator 106 determine that the multi-shock therapy is warranted, at least in part, based on the presence and/or capability of one or more devices in the vicinity of the subject 102 that are configured to administer the multi-shock therapy to the subject 102.

In various examples, it is preferred to administer the multi-shock therapy using coordinated electrical shocks delivered (e.g., output) by multiple devices. The primary defibrillator 106 is configured to output an electrical shock by discharging a primary capacitor. The primary capacitor, however, may be unable to carry enough charge to enable the primary defibrillator 106 to output multiple, sequential defibrillation-level electrical shocks by discharging the primary capacitor. While it may be possible to include multiple capacitors in the primary defibrillator 106, such additional circuitry can greatly increase the weight of the primary defibrillator 106. Thus, limiting the primary defibrillator 106 to a single capacitor for the purpose of defibrillation may enhance the portability of the primary defibrillator 106.

In some cases, it is additionally preferred to output the multiple electrical shocks of a multi-shock therapy at a specific timing relationship. Certain relative timing relationships between the electrical shocks can, in some cases, result in harm or ineffective treatments to the subject 102. For example, Taylor et al., July:140:194-200 (2019), which is incorporated by reference herein in its entirety, indicates that there is reduced efficacy for treating VF using electrical shocks applied at a 50 millisecond (ms) interval, compared to other timing relationships between applied electrical shocks.

Various implementations of the present disclosure address these and other issues by use of a multi-shock accessory 112. The multi-shock accessory 112, in various implementations, is configured to output an electrical shock coordinated with an electrical shock output by the primary defibrillator 106, such that both electrical shocks collectively form an effective multi-shock therapy for the subject 102. In various cases, the multi-shock accessory 112 is separate from the primary defibrillator 106. For example, the multi-shock accessory 112 has a separate housing from the primary defibrillator 106, such that the multi-shock accessory 112 can be transported independently from the primary defibrillator 106.

In various implementations, the multi-shock accessory 112 is coupled with secondary electrode pads 114 that are disposed on the chest of the subject 102. The multi-shock accessory 112, in various cases, includes a second treatment circuit that is electrically coupled with the secondary electrode pads 114. The second treatment circuit, for instance, includes a second capacitor. For example, the multi-shock accessory 112 is configured to output a secondary electrical shock by discharging the second capacitor to the secondary electrode pads 114. The inclusion of the second capacitor in the portable multi-shock accessory 112 may enable the multi-shock therapy while retaining the portability of the primary defibrillator 106 and the multi-shock accessory 112 in the environment 100.

In some implementations, the multi-shock accessory 112 is not a standalone defibrillator. For example, the multi-shock accessory 112 may omit one or more components of a standalone defibrillator, such as a display (e.g., a screen), input devices (e.g., buttons, ports, sensors, etc.), and output devices (e.g., speakers, lights, etc.). The omission of input devices and output devices, for example, may further reduce the weight of the multi-shock accessory 112, thereby enhancing its portability. In some cases, the omission of input devices and output devices within the multi-shock accessory 112 may prevent the rescuer 104 from being distracted during the rescue event. For example, the limited functionality of the multi-shock accessory 112 may enhance the usability of a multi-shock (e.g., DSD) system including the primary defibrillator 106 and the multi-shock accessory 112.

In some examples, the primary defibrillator 106 transmits a deactivation request to the multi-shock accessory 112 that causes the multi-shock accessory 112 to deactivate functionality of one or more features and/or accessories associated with the multi-shock accessory 112. For example, the multi-shock accessory 112 may deactivate a display, speaker, or other output device of the multi-shock accessory 112 in response to receiving the deactivation request. In some examples, the multi-shock accessory 112 includes one or more sensors (e.g., a measurement circuit configured to detect an ECG of the subject 102 via the secondary electrode pads 114) that are deactivated in response to the deactivation request. In some cases, the multi-shock accessory 112 deactivates an impedance-measuring functionality. For example, the multi-shock accessory 112 may be configured to detect a transthoracic impedance of the subject 102 by applying a periodic electrical signal between the secondary electrode pads 114. Upon receiving the deactivation request, the multi-shock accessory 112 may refrain from outputting the periodic electrical signal or may change a carrier frequency of the periodic electrical signal. Accordingly, the multi-shock accessory 112 may be prevented from distracting the rescuer 104 and/or internal circuitry of the multi-shock accessory 112 may be protected during administration of the multi-shock therapy.

In various cases, the primary defibrillator 106 coordinates timing of the delivery of a primary electrical shock and a secondary electrical shock in the multi-shock therapy by transmitting a multi-shock instruction 116 to the multi-shock accessory 112. The multi-shock instruction 116 is transmitted via a wired interface and/or a wireless interface, for instance. For instance, a cable may extend between the primary defibrillator 106 and the multi-shock accessory 112 along which a signal encoding the multi-shock instruction 116 is transmitted.

According to some examples, the primary defibrillator 106 and the multi-shock accessory 112 are configured to be wirelessly paired. Once paired, the primary defibrillator 106 is configured to transmit the multi-shock instruction 116 to the multi-shock accessory 112 over at least one wireless channel (also referred to as a “communication interface”).

In some implementations, the communication channel between the primary defibrillator 106 and the multi-shock accessory 112 is established when the primary defibrillator 106 and the multi-shock accessory 112 are paired. In particular cases, the primary defibrillator 106 and multi-shock accessory 112 refrain from sharing substantive data (e.g., physiological metrics, reports about the subject 102, instructions for treating the subject 102, etc.) until the primary defibrillator 106 and the multi-shock accessory 112 are paired. As used herein, the term “paired,” and its equivalents, may refer to a state of multiple devices that have a shared link key that enables each device to cryptographically authenticate data it receives from any other device among the multiple devices.

In particular cases, a first paired device encrypts data prior to transmitting the data to a second paired device, and the second paired device restores the original data by decrypting the encrypted data. As used herein, the term “encrypt,” and its equivalents, refers to a process of translating data from one format (e.g., an unencoded format) into an encoded format. In various cases, the encoded format is referred to as “ciphertext.” Unencoded data, which has not been encrypted, may be referred to as being in “plaintext.” In various examples, an entity encrypts data using at least one encryption key. An encryption key is a parameter that defines the translation of data from the one format into the encoded format. As used herein, the term “decrypt,” and its equivalents, refers to a process of translating data from an encoded format into another format (e.g., an unencoded format), such as a plaintext format. In various examples, an entity encrypts data using at least one decryption key. A decryption key is a parameter that defines the translation of data from the encoded format into the other format. A link key, for example, is an encryption and/or decryption key.

Various cryptographic techniques can be utilized in accordance with the features described in this disclosure. For example, data can be encrypted and decrypted via a symmetric key, wherein the encryption key and the decryption key are equivalent. In some cases, data can be encrypted and decrypted via asymmetric keys, wherein the encryption key and the decryption key are different. Cryptographic hash functions (CHFs) are examples of cryptographic techniques. Examples of cryptographic techniques include the Data Encryption Standard (DES), Advanced Encryption Standard (AES), Elliptic Curve Cryptography (ECC), Rivest-Shamir-Adleman (RSA), Secure Hash Algorithm (SHA)-1, SHA-2, SHA-3, BLAKE, BLAKE2, BLAKE3, WHIRLPOOL, MD2, MD4, MD5, MD6, Temporal Key Integrity Protocol (TKIP), Rivest cipher 4 (RC4), variably modified permutation composition (VMPC), blowfish, Twofish, Threefish, Tiny Encryption Algorithm (TEA), Extended TEA (XTEA), Corrected Block TEA (XXTEA), Diffie-Hellman exchange (DHE), elliptic curve DHE, supersingular isogeny Diffie-Hellman (SIDH) key exchange, and so on. Any suitable encryption or decryption technique can be used in accordance with implementations of this disclosure.

Various mechanisms can be utilized to pair the primary defibrillator 106 with the multi-shock accessory 112. For example, automated pairing may involve the exchange of data and/or pairing requests/responses between the primary defibrillator 106 and the multi-shock accessory 112. As another example, the primary defibrillator 106 may receive an input signal from an operator (e.g., the rescuer 104) that selects the multi-shock accessory 112 as a device to pair with the primary defibrillator 106, or vice versa. In some cases, the primary defibrillator 106 detects an alternative signal (e.g., a flashing light pattern) from the multi-shock accessory 112 that is indicated in a pairing request. In some cases, the primary defibrillator 106 and the multi-shock accessory 112 are paired, at least in part, based on signaling to and/or from an intermediary device (not illustrated). In a particular example, two or more devices can be brought into proximity to (e.g., into contact with) each other in order to facilitating pairing the primary defibrillator 106 with the multi-shock accessory 112. For example, a “tap-to-pair” functionality may allow a user to bring the primary defibrillator 106 (or a component thereof) into close proximity to (e.g., into contact with) the multi-shock accessory 112 (or a component thereof), or vice versa, and a short-range wireless protocol, such as BLUETOOTH, near-field communication (NFC), or the like, may be used to detect that the primary defibrillator 106 and multi-shock accessory 112 are within a threshold distance of each other, and, in response, the primary defibrillator 106 and the multi-shock accessory 112 may be paired. In some cases, an intermediary device, such as a phone, may be brought into close proximity to (e.g., into contact with) the primary defibrillator 106 and/or the multi-shock accessory 112 (e.g., by touching the intermediary device to both the primary defibrillator 106 and the multi-shock accessory 112 sequentially), and a short-range wireless protocol may be used to detect these proximity events involving the intermediary device, and, in response, the primary defibrillator 106 and the multi-shock accessory 112 may be paired.

In various cases, the primary defibrillator 106 and the multi-shock accessory 112 are paired when the multi-shock accessory 112 transmits an authentication request to the primary defibrillator 106 and the primary defibrillator 106 pairs with the multi-shock accessory 112 based on the authentication request. The first authentication request, according to some cases, is part of a handshake between the primary defibrillator 106 and the multi-shock accessory 112. As used herein, the term “handshake,” and its equivalents, refers to one or more signals transmitted between at least two endpoints that establish protocols of communication between the endpoints (e.g., prior to substantive data being exchanged between the endpoints). The primary defibrillator 106 and the multi-shock accessory 112, in some cases, utilize Transmission Control Protocol (TCP). TCP, for instance, utilizes handshakes to establish communication sessions between devices. For example, a first device transmits a synchronize message including a first sequence number to a second device. The second device, in turn, transmits a synchronize-acknowledgement message including a second sequence number as well as a first acknowledgement number that is one greater than the first sequence number. In response, the first device transmits an acknowledgement message including a second acknowledgment number that is one greater than the second sequence number to the second device. The first and second devices, in some examples, are paired once the first device transmits the acknowledgement message. The sequence and acknowledgement numbers are used to initialize counters that track messages and/or bytes of data transmitted between the paired devices.

In some examples, the authentication request includes or otherwise indicates a link key that enables the primary defibrillator 106 to pair with the multi-shock accessory 112. The multi-shock accessory 112, for instance, broadcasts the authentication request into the environment 100. In some examples, the multi-shock accessory 112 broadcasts the authentication request in response to receiving an input signal from the rescuer 104 (e.g., the rescuer has pushed a button on the multi-shock accessory 112), in response to being powered on, in response to initiating the treatment of the subject 102, or the like.

The multi-shock instruction 116, in various examples, causes the multi-shock accessory 112 to administer a secondary electrical shock across the secondary electrode pads 114. For example, the primary defibrillator 106 is configured to output a primary electrical shock across the primary electrode pads 108. In particular cases, the primary electrical shock and the secondary electrical shock collectively form a multi-shock therapy administered to the subject 102. For example, the primary electrical shock and the secondary electrical shock temporally overlap.

In various cases, the multi-shock instruction 116 includes an indication of a timing relationship between the primary electrical shock and the secondary electrical shock, and/or a time at which the multi-shock accessory 112 is instructed to output the secondary electrical shock in the multi-shock therapy. Based on the multi-shock instruction 116, the multi-shock accessory 112 may charge the secondary capacitor and/or output the secondary electrical shock by discharging the secondary capacitor at a particular time. In some implementations, the multi-shock instruction 116 causes the multi-shock accessory 112 to discharge the secondary capacitor in response to detecting a leading edge of the primary electrical shock output by the primary defibrillator 106. For instance, the multi-shock accessory 112 may detect the primary electrical shock by detecting an electrical signal from the secondary electrode pads 114. In some cases, the multi-shock instruction 116 causes the multi-shock accessory 112 to begin to discharge the secondary electrical shock at a predetermined delay period after detecting the leading edge of the primary electrical shock. Thus, in some cases, the multi-shock instruction 116 instructs the multi-shock accessory 112 to output the secondary electrical shock at a particular time and/or in response to detecting an event (e.g., the discharge of the primary electrical shock along the primary electrode pads 108).

During a pairing process, it may be beneficial for the primary defibrillator 106 to authenticate the multi-shock accessory 112 and/or for the multi-shock accessory 112 to authenticate the primary defibrillator 106. Various authentication mechanisms can be performed. For example, the pairing request includes an authentication request to the multi-shock accessory 112. If the multi-shock accessory 112 is an authentic accessory to be utilized with the primary defibrillator 106, the multi-shock accessory 112 transmits an authentication response based on the authentication request. The primary defibrillator 106, for instance, confirms that the multi-shock accessory 112 is authentic by analyzing the authentication response. In some cases, the primary defibrillator 106 confirms that the multi-shock accessory 112 by determining that the authentication response includes a code that is associated with an authentic device that is designed to be used with the primary defibrillator 106.

In particular cases, the authentication is performed based on a comparison of signals detected by the primary defibrillator 106 and the multi-shock accessory 112. For example, the authentication response may include an indication of an ECG detected by the multi-shock accessory 112 via the secondary electrode pads 114. The primary defibrillator 106 may confirm that the multi-shock accessory 112 is connected to the same subject 102 as the primary defibrillator 106 by determining that the ECG detected by the multi-shock accessory 112 shares one or more characteristics with the ECG detected by the primary defibrillator 106 via the primary electrode pads 108. For example, if the respective ECG signals include temporally aligned artifacts, arrhythmia characteristics, QRS complexes, spikes, or other features, then the primary defibrillator 106 may confirm that the multi-shock accessory 112 is connected to the subject 102. In various cases, the primary defibrillator 106 is configured to authenticate the multi-shock accessory 112 in response to detecting that the multi-shock accessory 112 is connected to the subject 102.

Optionally, the multi-shock accessory 112 is configured to be mechanically coupled with the primary defibrillator 106. In some cases, the multi-shock accessory 112 is configured to be removably coupled with the primary defibrillator 106. For instance, the primary defibrillator 106, in some cases, includes a docking mechanism that is configured to mechanically couple with a portion of the multi-shock accessory 112, thereby holding the multi-shock accessory 112 in a fixed position relative to the primary defibrillator 106. In some cases, the docking mechanism includes at least one electrical contact that electrically connects the primary defibrillator 106 to the multi-shock accessory 112 when the multi-shock accessory 112 is docked with the docking mechanism. Accordingly, various electrical signals described herein can be transmitted between the primary defibrillator 106 and the multi-shock accessory 112 via the docking mechanism.

In some cases, the multi-shock accessory 112 is, or is integrated into, a medical device. For example, the multi-shock accessory 112 may be integrated into a mechanical chest compression device, in some examples, or the mechanical chest compression device itself may be the multi-shock accessory 112. The secondary electrode pads 114, in some cases, are further integrated with the medical device. For instance, the secondary electrode pads 114 are integrated with a compressor (e.g., a plunger or chest-compression band) of the mechanical chest compression device and/or a backboard of the mechanical chest compression device. In various cases, the multi-shock accessory 112 is configured to administer chest compressions to the subject 102 by moving a plunger up-and-down on the chest of the subject 102, or by periodically tightening a band disposed around the chest of the subject 102. In various cases, the multi-shock accessory 112 includes a motor configured to move the plunger and/or tighten the band. The motor, for instance, is driven (e.g., powered) by an on-board energy source within the multi-shock accessory 112 and/or by a power source within the primary defibrillator 106. In various examples, one or both of the secondary electrode pads 114 are integrated with a backboard, cot, or other patient support apparatus on which the subject 102 is disposed. In some examples, the multi-shock accessory 112 and/or secondary electrode pads 114 are part of an implantable device and/or a wearable device.

In some examples, the multi-shock accessory 112 is integrated into an electrode pad. For example, the multi-shock accessory 112 may be integrated with at least one of the secondary electrode pads 114 that is disposed on the chest of the subject 102. In some cases, a cable connecting at least one of the secondary electrode pads 114 to the multi-shock accessory 112 is omitted. For instance, the secondary electrode pads 114 may share a common substrate that includes multiple electrodes, wherein the substrate is configured to be adhered to the chest of the subject 102. The multi-shock accessory 112, in various cases, is mounted on the common substrate. Thus, in some cases, the multi-shock accessory 112 integrated with the secondary electrode pads 114 can be applied to the chest of the subject 102 by the rescuer 104.

Although FIG. 1 illustrates that the secondary electrode pads 114 are connected to the multi-shock accessory 112, and not the primary defibrillator 106, implementations are not so limited. In some examples, the multi-shock accessory 112 is configured to be electrically coupled with the primary defibrillator 106, and the secondary electrode pads 114 are connected to the primary defibrillator 106. In various implementations, the multi-shock accessory 112 is configured to discharge the second capacitor to circuitry within the primary defibrillator 106, which provides the energy from the second capacitor as the secondary electrical shock output to the secondary electrode pads 114. In some examples, the multi-shock accessory 112 acts as a secondary treatment circuit that is configured to provide the energy for at least one sequential electrical shock output by the primary defibrillator 106.

In some cases, the multi-shock accessory 112 is a standalone device that facilitates communication between the primary defibrillator 106 and a secondary defibrillator (not illustrated). For example, the multi-shock accessory 112 is configured to communicate with the primary defibrillator 106 and the secondary defibrillator regarding timing and other parameters associated with multiple electrical shocks in a multi-shock therapy. In some examples, the secondary electrode pads 114 are connected to the secondary defibrillator. In particular cases, the primary defibrillator 106 is a monitor-defibrillator and the secondary defibrillator is an AED.

A particular example will now be described with reference to FIG. 1. The rescuer 104 arrives in the environment and connects the primary electrode pads 108 to the chest of the subject 102, who is experiencing cardiac arrest and has collapsed. The rescuer 104 connects the primary defibrillator 106 to the primary electrode pads 108. In various cases, the primary defibrillator 106 is configured to detect the ECG of the subject 102 via the primary electrode pads 108. The primary defibrillator 106 further detects that the ECG is indicative of VF, and outputs a recommendation to administer an initial electrical shock to the subject 102. Upon the rescuer 104 pressing the shock button 110, the primary defibrillator 106 discharges the primary capacitor to the primary electrode pads 108, thereby administering an electrical shock to the heart of the subject 102. In various cases, the primary defibrillator 106 detects that the ECG of the subject remains indicative of VF. For example, the primary defibrillator 106 determines that the subject 102 has a treatment-resistant shockable arrhythmia, such as refractory VF. In some cases, the primary defibrillator 106 determines (by analyzing the ECG) that the VF rhythm was temporarily resolved in response to the administration of the electrical shock, but that the heart of the subject 102 subsequently reentered VF (e.g., the subject 102 is determined to have recurrent VF). If the primary defibrillator 106 determines that the delay between the administration of the electrical shock and the recurrence of VF is below a threshold (e.g., in a range of 1 to 2 minutes), the primary defibrillator 106 may determine that the subject 102 has a treatment-resistant shockable arrhythmia.

While a separate rescuer is administering chest compressions to the subject 102, the rescuer 104 connects the secondary electrode pads 114 to the subject 102. Further, the rescuer 104 transports the multi-shock accessory 112 to the environment 100 and connects the secondary electrode pads 114 to the multi-shock accessory 112. Upon powering up the multi-shock accessory 112, the primary defibrillator 106 may automatically detect the presence of the multi-shock accessory 112 and pair with the multi-shock accessory 112. The multi-shock accessory 112, for instance, lacks a display or speaker, such that the rescuer 104 can ignore the multi-shock accessory 112 and focus on information provided by the primary defibrillator 106.

In some examples, the rescuer 104 triggers administration of a multi-shock therapy to the subject 102 using the primary defibrillator 106 and the multi-shock accessory 112. For example, the primary defibrillator 106 transmits the multi-shock instruction 116 to the multi-shock accessory 112 instructing the multi-shock accessory 112 to output a secondary electrical shock when the multi-shock accessory 112 detects administration of a primary electrical shock from the primary defibrillator 106. When the rescuer 104 presses the shock button 110, the primary defibrillator 106 discharges the primary electrical shock to the primary electrode pads 108. Further, the multi-shock accessory 112 discharges the secondary electrical shock to the secondary electrode pads 114, wherein the primary electrical shock and the secondary electrical shock are temporally overlapping (or partially overlapping, or separated by several milliseconds, or the like). In some cases, the primary defibrillator 106 determines that the ECG of the subject 102 is no longer indicative of VF after administration of the primary and secondary electrical shocks.

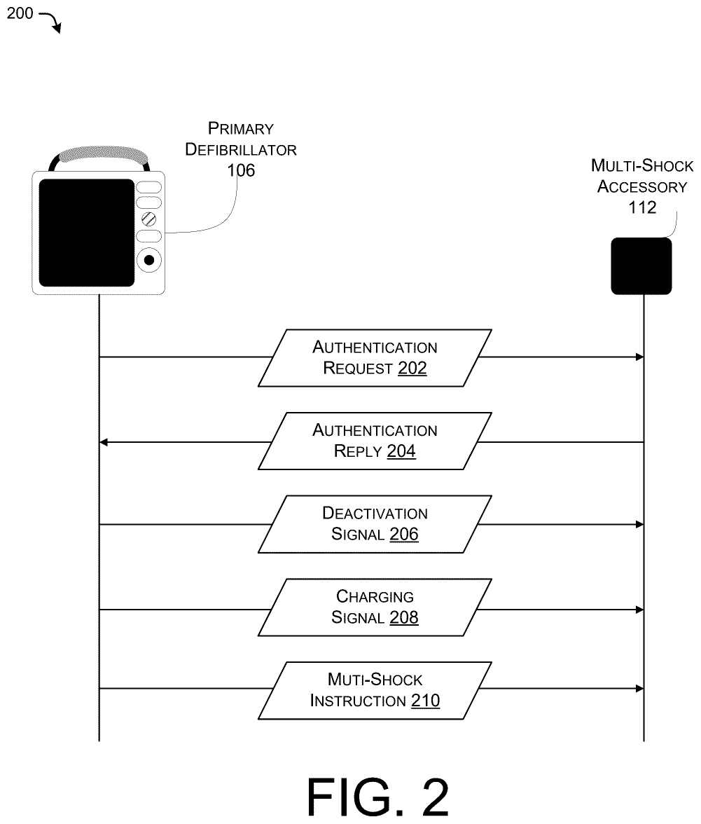

FIG. 2 illustrates example signaling 200 for coordinating a multi-shock therapy. The signaling 200, for instance, is between the primary defibrillator 106 and the multi-shock accessory 112 described above with reference to FIG. 1. The signaling 200 is transmitted over one or more wired interfaces, one or more wireless interfaces, or a combination thereof. In some examples, one or more communication signals in the signaling 200 are transmitted through the body of a subject that is electrically connected to the primary defibrillator 106 and the multi-shock accessory 112.

During an authentication process, the primary defibrillator 106 transmits an authentication request 202 to the multi-shock accessory 112. In various cases, the authentication request 202 is a pairing request in order to establish a communication channel between the primary defibrillator 106 and the multi-shock accessory 112. In some cases, the authentication request 202 includes a request for one or more authentication factors of the multi-shock accessory 112.

The multi-shock accessory 112 transmits an authentication reply 204 to the primary defibrillator 106. In various implementations, the authentication reply 204 is part of a pairing response that establishes the communication channel between the primary defibrillator 106 and the multi-shock accessory 112. In some cases, the authentication reply 204 includes one or more authentication factors of the multi-shock accessory 112. For example, the authentication reply 204 may include a code, a password, a software token, or some other data indicating a source and/or state of the multi-shock accessory 112. In some examples, the authentication reply 204 includes an indication of a manufacturer, a version, or model of the multi-shock accessory 112. In some cases, the authentication reply 204 includes an indication of a signal detected by the multi-shock accessory 112, such as a physiological parameter or signal detected from a subject that is being monitored by both the primary defibrillator 106 and the multi-shock accessory 112. In various cases, the primary defibrillator 106 authenticates the multi-shock accessory 112 and/or pairs with the multi-shock accessory 112 based on an analysis of the authentication reply 204. For example, the primary defibrillator 106 confirms that the one or more authentication factors match one or more criteria associated with an authentic device.

The primary defibrillator 106 transmits a deactivation signal 206 to the multi-shock accessory 112. Upon receiving the deactivation signal 206, the multi-shock accessory 112 deactivates one or more output devices (e.g., display, speaker, etc.) of the multi-shock accessory 112 and/or deactivates one or more input devices (e.g., physiological sensor, measurement circuit, buttons, touch sensors, etc.) of the multi-shock accessory 112. According to some cases, the multi-shock accessory 112 activates a single output device (e.g., a light source, display, etc.) indicating that the multi-shock accessory 112 is currently paired and/or coordinated with the primary defibrillator 106. For instance, the single output device indicates, to a user, that the multi-shock accessory 112 is not directly controllable by the user, at least temporarily. In some cases, the deactivation signal 206 is transmitted by the primary defibrillator 106 in response to the primary defibrillator 106 receiving the authentication reply 204.

The primary defibrillator 106 transmits a charging signal 208 to the multi-shock accessory 112. In various cases, the multi-shock accessory 112 charges a capacitor in response to receiving the charging signal 208. In various cases, the charging signal 208 is transmitted in response to the primary defibrillator 106 determining that a multi-shock therapy is indicated. In some cases, the charging signal 208 itself charges the capacitor. For instance, the charging signal 208 is an electrical signal that charges the capacitor. When the capacitor is charged, the multi-shock accessory 112 is prepared to output an electrical shock by discharging the capacitor. In some cases, the charging signal 208 indicates an energy level at which the capacitor is to be charged.

The primary defibrillator 106 transmits a multi-shock instruction 210 to the multi-shock accessory 112. In various cases, the multi-shock instruction 210 causes the multi-shock accessory 112 to output a secondary electrical shock. The multi-shock instruction 210, for instance, causes the multi-shock accessory 112 to output the secondary electrical shock having a predetermined timing relationship with a primary electrical shock output by the primary defibrillator 106. In some cases, the multi-shock instruction 210 causes the multi-shock accessory 112 to output the secondary electrical shock in response to detecting an event. For instance, the multi-shock accessory 112 may output the secondary electrical shock in response to detecting an electrical signal indicating that the primary defibrillator 106 has output the primary electrical shock. In some cases, the multi-shock instruction 210 indicates a vector at which the secondary shock is to be output and/or an energy level at which the secondary shock is to be output. In some examples, the multi-shock instruction 210 is the same as, or similar to, the multi-shock instruction 116 described above with reference to FIG. 1.

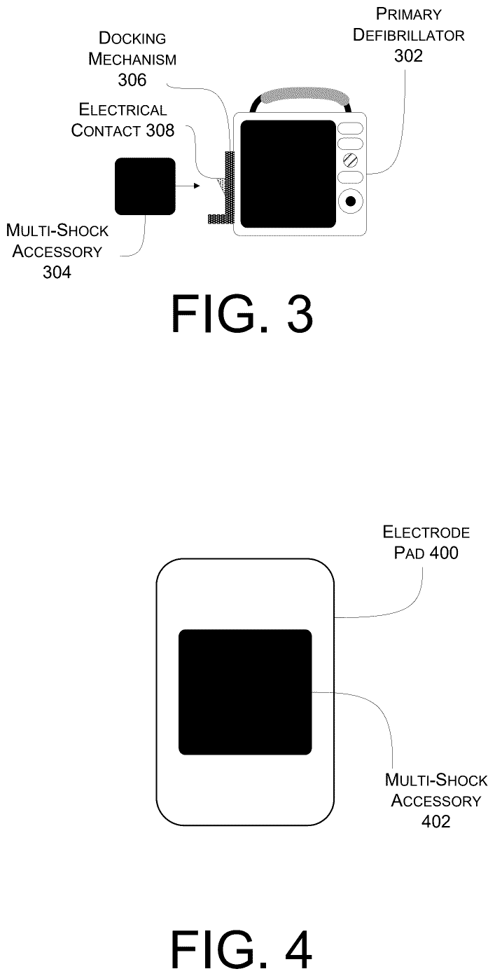

FIG. 3 illustrates an example of a primary defibrillator 302 configured to physically connect with a multi-shock accessory 304. For example, the primary defibrillator 302 is the primary defibrillator 106 and/or the multi-shock accessory 304 is the multi-shock accessory 112. In some cases, the multi-shock accessory 304 is a therapy delivery portion of the primary defibrillator 302 or of a secondary defibrillator, either of which could be a modular defibrillator.

The primary defibrillator 302 includes a docking mechanism 306 configured to physically couple with at least a portion of the multi-shock accessory 304. For example, the docking mechanism 306 includes a frame configured to conform to an outer surface of a housing of the multi-shock accessory 304. In some cases, the frame includes a base configured to be disposed underneath and to support a weight of the multi-shock accessory 304 when the multi-shock accessory 304 is coupled with the docking mechanism 306. In some examples, the frame includes a plate configured to conform to at least one side surface of the housing of the multi-shock accessory 304.

In various cases, the docking mechanism 306 includes a mechanical mechanism configured to hold the multi-shock accessory 304 in a predetermined position relative to the primary defibrillator 302. For example, the docking mechanism 306 includes one or more hooks configured to be inserted into one or more holes or concave portions of the housing of the multi-shock accessory 304, or vice versa. In some cases, the multi-shock accessory 304 can be decoupled from the docking mechanism 306 in response to a user selecting an input device (e.g., a button) of the primary defibrillator 302 and/or the docking mechanism 306.

When docked, the multi-shock accessory 304 is configured to be electrically connected with the primary defibrillator 302 via the docking mechanism 306. For example, the docking mechanism 306 includes an electrical contact 308 that is configured to be electrically connected to a circuit within the multi-shock accessory 304 when the multi-shock accessory 304 is physically coupled with the docking mechanism 306. In some cases, the electrical contact 308 is part of a hook of the docking mechanism 306. In various implementations, the multi-shock accessory 304 is configured to exchange signals with the primary defibrillator 302 via the electrical contact 308. For example, the multi-shock accessory 304 is configured to receive a communication signal with the primary defibrillator 302, to transmit a communication signal to the primary defibrillator 302, to receive energy from the primary defibrillator 302 (e.g., energy to charge a capacitor and/or battery of the multi-shock accessory 304), or the like. In various cases, the multi-shock accessory 304 is configured to charge a capacitor based on energy from the primary defibrillator 302 and/or based on energy from an on-board power source of the multi-shock accessory 304 (e.g., a battery in the multi-shock accessory 304).

In some alternative implementations, the multi-shock accessory 304 is configured to connect to the primary defibrillator 302 via an alternative docking mechanism. For instance, a cable extending from the multi-shock accessory 304 is configured to electrically connect to a proprietary receptacle in the primary defibrillator 302, providing at least one electrical and/or communication path between both devices.

FIG. 4 illustrates an example of an electrode pad 400 that is coupled with a multi-shock accessory 402. For example, the multi-shock accessory 402 is the multi-shock accessory 112 and/or the multi-shock accessory 304 discussed above. In various implementations, the electrode pad 400 includes one or more electrodes configured to be disposed on skin of a subject. In some cases, the electrode(s) are covered with an electrolyte gel to enhance an electrical connection between the electrode(s) and the skin. The electrode(s), for instance, are disposed on a first side of an electrically insulative substrate. The substrate, for instance, includes a polymer. In some cases, the first side of the electrically insulative substrate is at least partially coated with a biocompatible adhesive configured to maintain the electrical connection between the electrode(s) and the skin.

In some implementations, the multi-shock accessory 402 is disposed on a second side of the electrically insulative substrate. For example, the multi-shock accessory 402 is configured to be electrically connected with the electrode(s) disposed on the opposite side of the electrode pad 400. In some cases, at least one of the electrode(s) are configured to be connected to with a primary defibrillator. The multi-shock accessory 112 and/or the primary defibrillator are configured to output at least one electrical shock to the electrode(s) of the electrode pad 400. In some cases, the electrode pad 400, including the multi-shock accessory 402, is a disposable device. In some cases, the electrode pad 400 is disposable and configured to be removably coupled with the multi-shock accessory 402, which may be reused with other electrode pads.

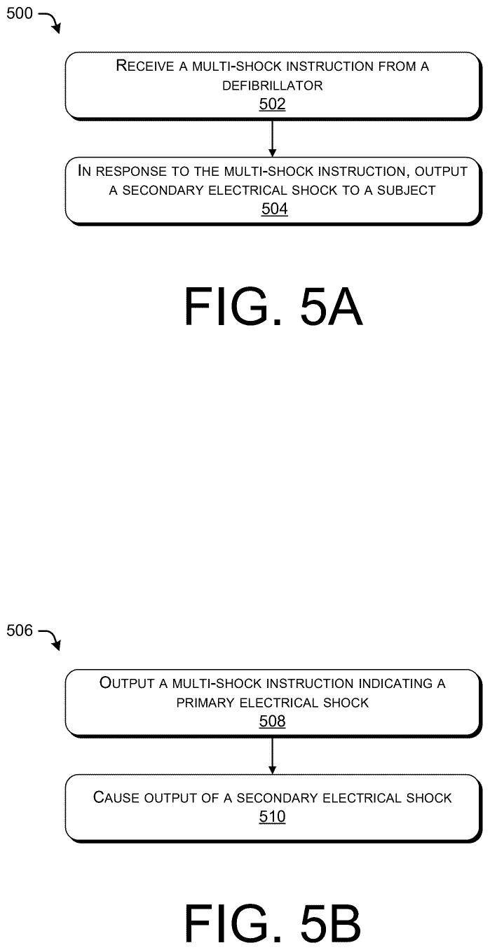

FIG. 5A illustrates an example process 500 for administering a multi-shock therapy based on an instruction from a defibrillator. The process 500 is performed by an entity including, for example, at least one of the multi-shock accessory 112, the multi-shock accessory 304, the multi-shock accessory 402, a defibrillator, a mechanical chest compression device, or a medical device.

At 502, the entity receives a multi-shock instruction from a defibrillator. The multi-shock instruction, for instance, indicates a scheduled time of one or more electrical shocks in a planned multi-shock therapy. For example, the multi-shock instruction indicates the time of a primary electrical shock or a secondary electrical shock. In some cases, the entity receives a charging signal from the defibrillator. The charging signal, for instance, causes the entity to charge a capacitor using one or more power sources (e.g., batteries). In some implementations, the charging signal is part of the multi-shock instruction or part of a separate communication signal from the multi-shock instruction, for instance. In various cases, the multi-shock instruction indicates a vector at which the secondary electrical shock is to be output. In some examples, the multi-shock instruction and/or charging signal indicates an energy level at which the secondary shock is to be administered. In some cases, the charging signal itself is an electrical signal configured to charge the capacitor of the entity.

At 504, in response to the multi-shock instruction, the entity outputs a secondary electrical shock to a subject. The secondary electrical shock, for instance, temporally overlaps the primary electrical shock. In some cases, the secondary electrical shock and the primary electrical shock are sequential. In various cases, the entity includes a treatment circuit configured to output the secondary electrical shock to electrodes disposed on the skin of a subject. In some cases, the entity confirms that the electrodes are disposed along the vector indicated in the multi-shock instruction. In various cases, the entity outputs the secondary electrical shock to the electrodes by discharging the capacitor. In various examples, the entity outputs the secondary electrical shock at the energy level specified in the multi-shock instruction and/or charging signal.

In some cases, the entity is configured to detect an ECG from the electrodes. In various cases, the entity is configured to remove, from the ECG, a chest compression artifact from the ECG. For example, the entity may administer the chest compressions and remove the chest compression artifact associated with a time and/or frequency at which the chest compressions are administered to the subject. For instance, the entity may apply a comb filter to the ECG, wherein the comb filter rejects a frequency of the chest compressions and one or more harmonics of the frequency. In some cases, the entity utilizes the same power source (e.g., battery) for driving a motor that causes the entity to administer the chest compressions and for charging the capacitor.

FIG. 5B illustrates an example process 506 for coordinating a multi-shock therapy with defibrillator. The process 506 is performed by an entity including, for example, at least one of primary defibrillator 106, the primary defibrillator 302, a defibrillator, a computing device, at least one processor, or a medical device.

At 508, the entity outputs a multi-shock instruction indicating a primary electrical shock. For example, the multi-shock instruction is output to a primary defibrillator and/or to a multi-shock accessory. In various cases, the multi-shock instruction causes the primary defibrillator to output a primary electrical shock at a particular time. The primary electrical shock, for instance, is output via first electrodes disposed on a skin of a subject. In some cases, the multi-shock instruction causes the primary defibrillator and/or the multi-shock accessory to charge one or more capacitors in advance of administering electrical shocks to the subject. In some examples, the entity outputs the multi-shock instruction in response to receiving a user input signal. The multi-shock instruction, in some implementations, indicates a vector of the primary electrical shock and/or a vector of the secondary electrical shock. In various cases, the multi-shock instruction indicates an energy level of the primary electrical shock and/or an energy level of the secondary electrical shock.

At 510, the entity causes output of a secondary electrical shock. In some cases, the entity itself is a multi-shock accessory and outputs the secondary electrical shock to second electrodes disposed on the skin of the subject. The first electrodes and the second electrodes may be disposed at different vectors. In some examples, the entity instructs the multi-shock accessory to output the secondary electrical shock. The secondary electrical shock, in various cases, temporally overlaps with the primary electrical shock. In some cases, the primary electrical shock and the secondary electrical shock are sequential.

In some cases, the entity is configured to detect an ECG from the second electrodes. In various cases, the entity is configured to remove, from the ECG, a chest compression artifact from the ECG. For example, the entity may administer the chest compressions and remove the chest compression artifact associated with a time and/or frequency at which the chest compressions are administered to the subject. For instance, the entity may apply a comb filter to the ECG, wherein the comb filter rejects a frequency of the chest compressions and one or more harmonics of the frequency. In some cases, the entity utilizes the same power source (e.g., battery) for driving a motor that causes the entity to administer the chest compressions and for charging the capacitor.

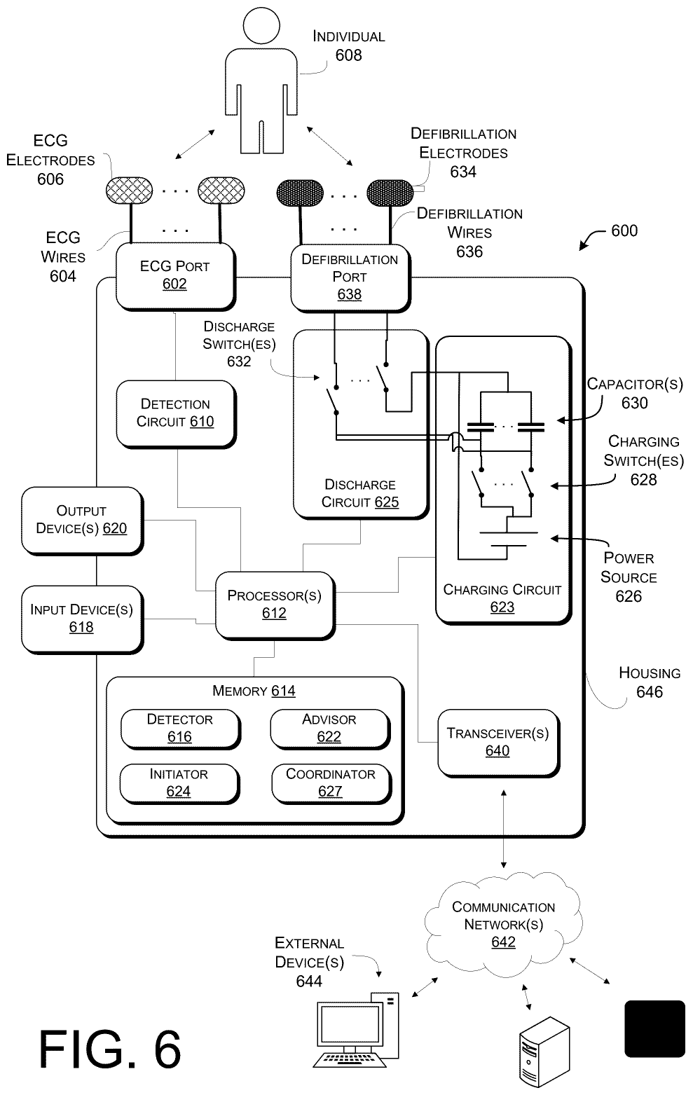

FIG. 6 illustrates an example of an external defibrillator 600 configured to perform various functions described herein. For example, the external defibrillator 600 is the primary defibrillator 106 and/or the multi-shock accessory 112 described above with reference to FIG. 1.

The external defibrillator 600 includes an electrocardiogram (ECG) port 602 connected to multiple ECG wires 604. In some cases, the ECG wires 604 are removeable from the ECG port 602. For instance, the ECG wires 604 are plugged into the ECG port 602 via connectors. The ECG wires 604 are connected to ECG electrodes 606, respectively. In various implementations, the ECG electrodes 606 are disposed on different locations on an individual 608. A detection circuit 610 (also referred to as a “measurement circuit”) is configured to detect relative voltages between the ECG electrodes 606. These voltages are indicative of the electrical activity of the heart of the individual 608.

In various implementations, the ECG electrodes 606 are in contact with the different locations on the skin of the individual 608. In some examples, a first one of the ECG electrodes 606 is placed on the skin between the heart and right arm of the individual 608, a second one of the ECG electrodes 606 is placed on the skin between the heart and left arm of the individual 608, and a third one of the ECG electrodes 606 is placed on the skin between the heart and a leg (either the left leg or the right leg) of the individual 608. In these examples, the detection circuit 610 is configured to measure the relative voltages between the first, second, and third ECG electrodes 606. Respective pairings of the ECG electrodes 606 are referred to as “leads,” and the voltages between the pairs of ECG electrodes 606 are known as “lead voltages.” In some examples, more than three ECG electrodes 606 are included, such that 5-lead or 12-lead ECG signals are detected by the detection circuit 610.

The detection circuit 610 includes at least one analog circuit, at least one digital circuit, or a combination thereof. The detection circuit 610 receives the analog electrical signals from the ECG electrodes 606, via the ECG port 602 and the ECG wires 604. In some cases, the detection circuit 610 includes one or more analog filters configured to filter noise and/or artifact from the electrical signals. The detection circuit 610 includes an analog-to-digital (ADC) in various examples. The detection circuit 610 generates a digital signal indicative of the analog electrical signals from the ECG electrodes 606. This digital signal can be referred to as an “ECG signal” or an “ECG.”

In some cases, the detection circuit 610 further detects an electrical impedance between at least one pair of the ECG electrodes 606. For example, the detection circuit 610 includes, or otherwise controls, a power source that applies a known voltage (or current) across a pair of the ECG electrodes 606 and detects a resultant current (or voltage) between the pair of the ECG electrodes 606. In various cases, the current is applied via a high-frequency (e.g., 20 kHz) carrier signal. The impedance is generated based on the applied signal (voltage or current) and the resultant signal (current or voltage). In various cases, the impedance corresponds to respiration of the individual 608, chest compressions performed on the individual 608, and other physiological states of the individual 608. In various examples, the detection circuit 610 includes one or more analog filters configured to filter noise and/or artifact from the resultant signal. The detection circuit 610 generates a digital signal indicative of the impedance using an ADC. This digital signal can be referred to as an “impedance signal” or an “impedance.”

The detection circuit 610 provides the ECG signal and/or the impedance signal one or more processors 612 in the external defibrillator 600. In some implementations, the processor(s) 612 includes a central processing unit (CPU), a graphics processing unit (GPU), both CPU and GPU, or other processing unit or component known in the art.

The processor(s) 612 is operably connected to memory 614. In various implementations, the memory 614 is volatile (such as random access memory (RAM)), non-volatile (such as read only memory (ROM), flash memory, etc.) or some combination of the two. The memory 614 stores instructions that, when executed by the processor(s) 612, causes the processor(s) 612 to perform various operations. In various examples, the memory 614 stores methods, threads, processes, applications, objects, modules, any other sort of executable instruction, or a combination thereof. In some cases, the memory 614 stores files, databases, or a combination thereof. In some examples, the memory 614 includes, but is not limited to, RAM, ROM, electrically erasable programmable read-only memory (EEPROM), flash memory, or any other memory technology. In some examples, the memory 614 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 612 and/or the external defibrillator 600. In some cases, the memory 614 at least temporarily stores the ECG signal and/or the impedance signal.

In various examples, the memory 614 includes a detector 616, which causes the processor(s) 612 to determine, based on the ECG signal and/or the impedance signal, whether the individual 608 is exhibiting a particular heart rhythm. For instance, the processor(s) 612 determines whether the individual 608 is experiencing a shockable rhythm that is treatable by defibrillation. Examples of shockable rhythms include ventricular fibrillation (VF) and ventricular tachycardia (V-Tach). In some examples, the processor(s) 612 determines whether any of a variety of different rhythms (e.g., asystole, sinus rhythm, atrial fibrillation (AF), etc.) are present in the ECG signal.

The processor(s) 612 is operably connected to one or more input devices 618 and one or more output devices 620. Collectively, the input device(s) 618 and the output device(s) 620 function as an interface between a user and the defibrillator 600. The input device(s) 618 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 620 includes at least one of a display, a speaker, a haptic output device, a printer, or any combination thereof. In various examples, the processor(s) 612 causes a display among the input device(s) 618 to visually output a waveform of the ECG signal and/or the impedance signal. In some implementations, the input device(s) 618 includes one or more touch sensors, the output device(s) 620 includes a display screen, and the touch sensor(s) are integrated with the display screen. Thus, in some cases, the external defibrillator 600 includes a touchscreen configured to receive user input signal(s) and visually output physiological parameters, such as the ECG signal and/or the impedance signal.

In some examples, the memory 614 includes an advisor 622, which, when executed by the processor(s) 612, causes the processor(s) 612 to generate advice and/or control the output device(s) 620 to output the advice to a user (e.g., a rescuer). In some examples, the processor(s) 612 provides, or causes the output device(s) 620 to provide, an instruction to perform CPR on the individual 608. In some cases, the processor(s) 612 evaluates, based on the ECG signal, the impedance signal, or other physiological parameters, CPR being performed on the individual 608 and causes the output device(s) 620 to provide feedback about the CPR in the instruction. According to some examples, the processor(s) 612, upon identifying that a shockable rhythm is present in the ECG signal, causes the output device(s) 620 to output an instruction and/or recommendation to administer a defibrillation shock to the individual 608.

The memory 614 also includes an initiator 624 which, when executed by the processor(s) 612, causes the processor(s) 612 to control other elements of the external defibrillator 600 in order to administer a defibrillation shock to the individual 608. In some examples, the processor(s) 612 executing the initiator 624 selectively causes the administration of the defibrillation shock based on determining that the individual 608 is exhibiting the shockable rhythm and/or based on an input from a user (received, e.g., by the input device(s) 618. In some cases, the processor(s) 612 causes the defibrillation shock to be output at a particular time, which is determined by the processor(s) 612 based on the ECG signal and/or the impedance signal.

In various cases, the memory 614 includes a coordinator 627 configured to coordinate administration of a multi-shock therapy with a separate device. In some cases, the coordinator 627, when executed by the processor(s) 612, causes the defibrillator 600 to pair with and/or authenticate the separate device, to send a deactivation signal, to deactivate one or more elements of the defibrillator 600 in response to a deactivation signal, to send a charging signal, to charge a capacitor in response to a charging signal, to send a multi-shock instruction, to output a primary electrical shock, to output a secondary electrical shock, or a combination thereof.

The processor(s) 612 is operably connected to a charging circuit 623 and a discharge circuit 625. In various implementations, the charging circuit 623 includes a power source 626, one or more charging switches 628, and one or more capacitors 630. The power source 626 includes, for instance, a battery. The processor(s) 612 initiates a defibrillation shock by causing the power source 626 to charge at least one capacitor among the capacitor(s) 630. For example, the processor(s) 612 activates at least one of the charging switch(es) 628 in the charging circuit 623 to complete a first circuit connecting the power source 626 and the capacitor to be charged. Then, the processor(s) 612 causes the discharge circuit 625 to discharge energy stored in the charged capacitor across a pair of defibrillation electrodes 634, which are in contact with the individual 608. For example, the processor(s) 612 deactivates the charging switch(es) 628 completing the first circuit between the capacitor(s) 630 and the power source 626, and activates one or more discharge switches 632 completing a second circuit connecting the charged capacitor 630 and at least a portion of the individual 608 disposed between defibrillation electrodes 634. In various cases, more than two defibrillation electrodes 634 are disposed on the skin of the individual 607, which define multiple shock vectors.

The energy is discharged from the defibrillation electrodes 634 in the form of a defibrillation shock. For example, the defibrillation electrodes 634 are connected to the skin of the individual 608 and located at positions on different sides of the heart of the individual 608, such that the defibrillation shock is applied across the heart of the individual 608. The defibrillation shock, in various examples, depolarizes a significant number of heart cells in a short amount of time. The defibrillation shock, for example, interrupts the propagation of the shockable rhythm (e.g., VF or VT) through the heart. In some examples, the defibrillation shock is 200 J or greater with a duration of about 0.015 seconds. In some cases, the defibrillation shock has a multiphasic (e.g., biphasic) waveform. The discharge switch(es) 632 are controlled by the processor(s) 612, for example. In various implementations, the defibrillation electrodes 634 are connected to defibrillation leads 636. The defibrillation wires 636 are connected to a defibrillation port 638, in implementations. According to various examples, the defibrillation wires 636 are removable from the defibrillation port 638. For example, the defibrillation wires 636 are plugged into the defibrillation port 638.

In various implementations, the processor(s) 612 is operably connected to one or more transceivers 640 that transmit and/or receive data over one or more communication networks 642. For example, the transceiver(s) 640 includes a network interface card (NIC), a network adapter, a local area network (LAN) adapter, or a physical, virtual, or logical address to connect to the various external devices and/or systems. In various examples, the transceiver(s) 640 includes any sort of wireless transceivers capable of engaging in wireless communication (e.g., radio frequency (RF) communication). For example, the communication network(s) 642 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 640 includes other wireless modems, such as a modem for engaging in WI-FI®, WIGIG®, WIMAX®, BLUETOOTH®, or infrared communication over the communication network(s) 642.

The defibrillator 600 is configured to transmit and/or receive data (e.g., ECG data, impedance data, data indicative of one or more detected heart rhythms of the individual 608, data indicative of one or more defibrillation shocks administered to the individual 608, etc.) with one or more external devices 644 via the communication network(s) 642. The external devices 644 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 642. In some examples, the external device(s) 644 is located remotely from the defibrillator 600, such as at a remote clinical environment (e.g., a hospital). According to various implementations, the processor(s) 612 causes the transceiver(s) 640 to transmit data to the external device(s) 644. In some cases, the transceiver(s) 640 receives data from the external device(s) 644 and the transceiver(s) 640 provide the received data to the processor(s) 612 for further analysis.

In various implementations, the external defibrillator 600 also includes a housing 646 that at least partially encloses other elements of the external defibrillator 600. For example, the housing 646 encloses the detection circuit 610, the processor(s) 612, the memory 614, the charging circuit 623, the transceiver(s) 640, or any combination thereof. In some cases, the input device(s) 618 and output device(s) 620 extend from an interior space at least partially surrounded by the housing 646 through a wall of the housing 646. In various examples, the housing 646 acts as a barrier to moisture, electrical interference, and/or dust, thereby protecting various components in the external defibrillator 600 from damage.

In some implementations, the external defibrillator 600 is an automated external defibrillator (AED) operated by an untrained user (e.g., a bystander, layperson, etc.) and can be operated in an automatic mode. In automatic mode, the processor(s) 612 automatically identifies a rhythm in the ECG signal, makes a decision whether to administer a defibrillation shock, charges the capacitor(s) 630, discharges the capacitor(s) 630, or any combination thereof. In some cases, the processor(s) 612 controls the output device(s) 620 to output (e.g., display) a simplified user interface to the untrained user. For example, the processor(s) 612 refrains from causing the output device(s) 620 to display a waveform of the ECG signal and/or the impedance signal to the untrained user, in order to simplify operation of the external defibrillator 600.

In some examples, the external defibrillator 600 is a monitor-defibrillator utilized by a trained user (e.g., a clinician, an emergency responder, etc.) and can be operated in a manual mode or the automatic mode. When the external defibrillator 600 operates in manual mode, the processor(s) 612 cause the output device(s) 620 to display a variety of information that may be relevant to the trained user, such as waveforms indicating the ECG data and/or impedance data, notifications about detected heart rhythms, and the like.

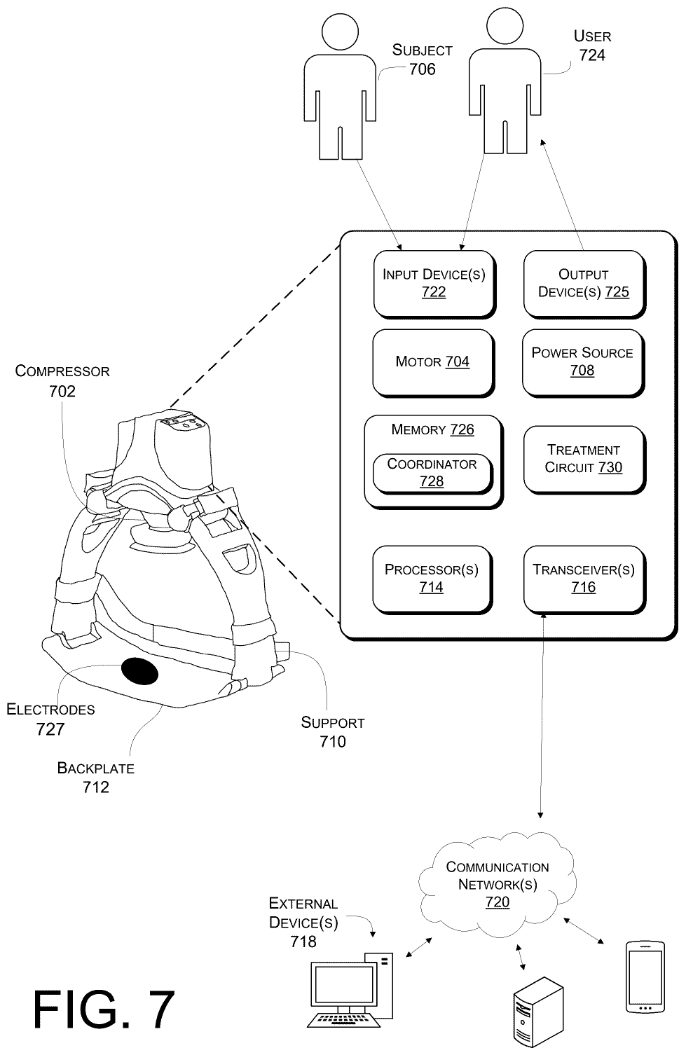

FIG. 7 illustrates a chest compression device 700 configured to perform various functions described herein. For example, the chest compression device 700 is the multi-shock accessory 112 described in FIG. 1.

In various implementations, the chest compression device 700 includes a compressor 702 that is operatively coupled to a motor 704. The compressor 702 physically administers a force to the chest of a subject 706 that compresses the chest of the subject 706. In some examples, the compressor 702 includes at least one piston that periodically moves between two positions (e.g., a compressed position and a release position) at a compression frequency. For example, when the piston is positioned on the chest of the subject 706, the piston compresses the chest when the piston is moved into the compressed position. A suction cup may be positioned on a tip of the piston, such that the suction cup contacts the chest of the subject 706 during operation. In various cases, the compressor 702 includes a band that periodically tightens to a first tension and loosens to a second tension at a compression frequency. For instance, when the band is disposed around the chest of the subject 706, the band compresses the chest when the band tightens.

The motor 704 is configured to convert electrical energy stored in a power source 708 into mechanical energy that moves and/or tightens the compressor 702, thereby causing the compressor 702 to administer the force to the chest of the subject 706. In various implementations, the power source 708 is portable. For instance, the power source 708 includes at least one rechargeable (e.g., lithium-ion) battery. In some cases, the power source 708 supplies electrical energy to one or more elements of the chest compression device 700 described herein.

In various cases, the chest compression device 700 includes a support 710 that is physically coupled to the compressor 702, such that the compressor 702 maintains a position relative to the subject 706 during operation. In some implementations, the support 710 is physically coupled to a backplate 712, cot, or other external structure with a fixed position relative to the subject 706. According to some cases, the support 710 is physically coupled to a portion of the subject 706, such as wrists of the subject 706.

The operation of the chest compression device 700 may be controlled by at least one processor 714. In various implementations, the motor 704 is communicatively coupled to the processor(s) 714. Specifically, the processor(s) 714 is configured to output a control signal to the motor 704 that causes the motor 704 to actuate the compressor 702. For instance, the motor 704 causes the compressor 702 to administer the compressions to the subject 706 based on the control signal. In some cases, the control signal indicates one or more treatment parameters of the compressions. Examples of treatment parameters include a frequency, timing, depth, force, position, velocity, and acceleration of the compressor 702 administering the compressions. According to various cases, the control signal causes the motor 704 to cease compressions.

In various implementations, the chest compression device 700 includes at least one transceiver 716 configured to communicate with at least one external device 718 over one or more communication networks 720. Any communication network described herein can be included in the communication network(s) 720 illustrated in FIG. 7. The external device(s) 718, for example, includes at least one of a monitor-defibrillator, an AED, an ECMO device, a ventilation device, a patient monitor, a mobile phone, a server, or a computing device. In some implementations, the transceiver(s) 716 is configured to communicate with the external device(s) 718 by transmitting and/or receiving signals wirelessly. For example, the transceiver(s) 716 includes a NIC, a network adapter, a LAN adapter, or a physical, virtual, or logical address to connect to the various external devices and/or systems. In various examples, the transceiver(s) 716 includes any sort of wireless transceivers capable of engaging in wireless communication (e.g., RF communication). For example, the communication network(s) 720 includes one or more wireless networks that include a 3GPP network, such as an LTE RAN (e.g., over one or more LTE bands), an NR RAN (e.g., over one or more NR bands), or a combination thereof. In some cases, the transceiver(s) 716 includes other wireless modems, such as a modem for engaging in WI-FI®, WIGIG®, WIMAX®, BLUETOOTH®, or infrared communication over the communication network(s) 720. The signals, in various cases, encode data in the form of data packets, datagrams, or the like. In some cases, the signals are transmitted as compressions are being administered by the chest compression device 700 (e.g., for real-time feedback by the external device(s) 718), after compressions are administered by the chest compression device 700 (e.g., for post-event review at the external device 718), or a combination thereof.

In various cases, the processor(s) 714 generates the control signal based on data encoded in the signals received from the external device(s) 718. For instance, the signals include an instruction to initiate the compressions, and the processor(s) 714 instructs the motor 704 to begin actuating the compressor 702 in accordance with the signals.

In some cases, the chest compression device 700 includes at least one input device 722. In various examples, the input device(s) 722 is configured to receive an input signal from a user 724, who may be a rescuer treating the subject 706. Examples of the input device(s) 722 include, for instance, at a keypad, a cursor control, a touch-sensitive display, a voice input device (e.g., a microphone), a haptic feedback device (e.g., a gyroscope), or any combination thereof. In various implementations, the processor(s) 714 generate the control signal based on the input signal. For instance, the processor(s) 714 generate the control signal to adjust a frequency of the compressions based on the chest compression device 700 detecting a selection by the user 724 of a user interface element displayed on a touchscreen or detecting the user 724 pressing a button integrated with an external housing of the chest compression device 700.

According to some examples, the input device(s) 722 include one or more sensors. The sensor(s), for example, is configured to detect a physiological parameter of the subject 706. In some implementations, the sensor(s) is configured to detect a state parameter of the chest compression device 700, such as a position of the compressor 702 with respect to the subject 706 or the backplate 712, a force administered by the compressor 702 on the subject 706, a force administered onto the backplate 712 by the body of the subject 706 during a compression, or the like. According to some implementations, the signals transmitted by the transceiver(s) 716 indicate the physiological parameter(s) and/or the state parameter(s).

The chest compression device 700 further includes at least one output device 725, in various implementations. Examples of the output device(s) 725 include, for instance, least one of a display (e.g., a projector, an LED screen, etc.), a speaker, a haptic output device, a printer, or any combination thereof. In some implementations, the output device(s) 725 include a screen configured to display various parameters detected by and/or reported to the chest compression device 700, a charge level of the power source 708, a timer indicating a time since compressions were initiated or paused, and other relevant information.

The chest compression device 700 further includes memory 726. In various implementations, the memory 726 is volatile (such as random access memory (RAM)), non-volatile (such as read only memory (ROM), flash memory, etc.) or some combination of the two. The memory 726 stores instructions that, when executed by the processor(s) 714, causes the processor(s) 714 to perform various operations. In various examples, the memory 726 stores methods, threads, processes, applications, objects, modules, any other sort of executable instruction, or a combination thereof. In some cases, the memory 726 stores files, databases, or a combination thereof. In some examples, the memory 726 includes, but is not limited to, RAM, ROM, EEPROM, flash memory, or any other memory technology. In some examples, the memory 726 includes one or more of CD-ROMs, DVDs, CAM, or other optical storage, magnetic cassettes, magnetic tape, magnetic disk storage or other magnetic storage devices, or any other medium which can be used to store the desired information. In various cases, the memory 726 stores instructions, programs, threads, objects, data, or any combination thereof, that cause the processor(s) 714 to perform various functions. In various cases, the memory 726 stores one or more parameters that are detected by the chest compression device 700 and/or reported to the chest compression device 700.

In implementations of the present disclosure, the chest compression device 700 includes electrodes 727 configured to output an electrical shock to the subject 706 when the subject 706 is positioned on the backplate 712. For instance, the electrodes 727 are disposed on surfaces of the backplate 712, the compressor 702, or a combination thereof. In some examples, the memory 726 also stores instructions for executing a coordinator 728. When executed by the processor(s) 714, for instance, the coordinator 728 causes the chest compression device 700 to transmit a pairing response and/or an authentication response, to deactivate one or more elements of the chest compression device 700, to charge a capacitor of a treatment circuit 730 in the chest compression device 700, to administer a secondary electrical shock by discharging the capacitor of the treatment circuit 730 to the electrodes 727, or various other functions described herein. In some cases, the capacitor of the treatment circuit 730 is charged using the power source 708.

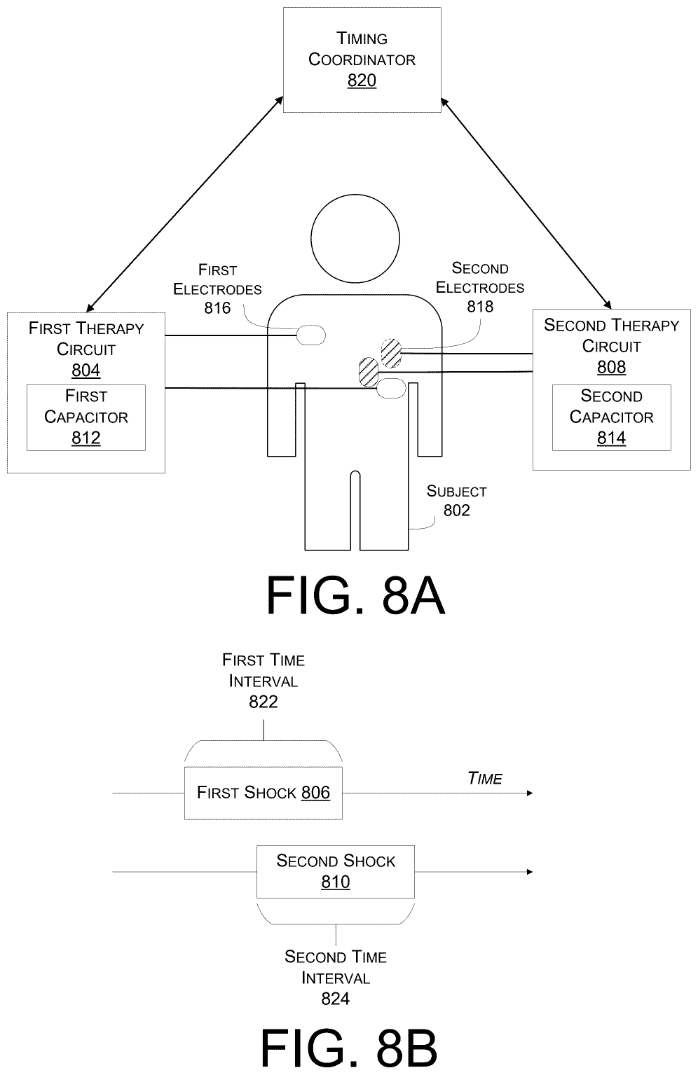

FIGS. 8A and 8B illustrate examples of environments and timing related to administering a multi-shock therapy (e.g., a DSD therapy). FIG. 8A shows an environment configured to administer the multi-shock therapy. FIG. 8B shows a timing relationship of multiple shocks administered in the multi-shock therapy.

In various cases, a subject 802 has a medical condition that is treatable by defibrillation. For example, the subject 802 may have a shockable cardiac arrhythmia, such as VF or pulseless VT. In some cases, however, conventional defibrillation therapies do not resolve the medical condition. For example, the subject 802 may be experiencing VF that does not resolve after the administration of a single biphasic electrical shock administered by a defibrillator. For instance, the subject 802 may have refractory VF.

In various implementations of the present disclosure, the medical condition of the subject 802 is treatable by administration of a multi-shock therapy. Specifically, a first therapy circuit 804 is configured to output a first shock 806 to the subject 802 and a second therapy circuit 808 is configured to output a second shock 810 to the subject 802. In various cases, the first shock 806 and the second shock 810 temporally overlap in time, at least partially. For example, a start time of the second shock 810 occurs after the start time of the first shock 806, but the start time of the second shock 810 occurs before the end time of the first shock 806. In various cases, the first shock 806 is a biphasic shock and/or the second shock 810 is a biphasic shock. In some implementations, the first shock 806 is a monophasic shock and/or the second shock 810 is a biphasic shock. In some cases, the first shock 806 has a shorter duration and/or lower voltage amplitude than the second shock 810.

The first therapy circuit 804 outputs the first shock 806 by discharging a first capacitor 812. Similarly, the second therapy circuit 808 outputs the second shock 810 by discharging a second capacitor 814. In various implementations, one or more power sources are configured to charge the first capacitor 812 and/or the second capacitor 814 prior to discharge. In various cases, the first therapy circuit 804 includes a first H-bridge circuit including the first capacitor 812 and/or the second therapy circuit 808 includes a second H-bridge circuit including the second capacitor 814. The first H-bridge circuit and the second H-bridge circuit are configured to output the first shock 806 and the second shock 810 as biphasic shocks, for instance, via sequential activation of switches in the first H-bridge circuit and the second H-bridge circuit.

The first therapy circuit 804 is configured to output the first shock 806 to first electrodes 816. The second therapy circuit 808 is configured to output the second shock 810 to second electrodes 818. In various cases, the first electrodes 816 and/or the second electrodes 818 are disposed externally on the skin of the subject 802. For example, the first electrodes 816 and/or the second electrodes 818 are adhered to the skin of the subject 802. In various implementations, the first electrodes 816 and the second electrodes 818 are associated with different shock vectors. For instance, a first shock vector extends between the first electrodes 816 and a second shock vector extends between the second electrodes 818, wherein the first shock vector and the second shock vector are different. For example, the first shock vector may be an anterior-lateral position and the second shock vector may be an anterior-posterior position. In various implementations, the first shock vector and the second shock vector both extend through the heart of the subject 802. Although FIG. 8A illustrates the first electrodes 816 as being separate from the second electrodes 818, implementations are not so limited. For example, one electrode may be shared among the first electrodes 816 and the second electrodes 818. In various cases, both the first shock vector and the second shock vector are optimal vectors, as described elsewhere herein.

The first therapy circuit 804 and the second therapy circuit 808 are distributed among one or more devices. In some cases, the first therapy circuit 804 is part of a first external defibrillator and the second therapy circuit 808 is part of a second external defibrillator. For example, the first external defibrillator and the second external defibrillator are both monitor-defibrillators, both AEDs, or a monitor-defibrillator and an AED. In some cases, the first therapy circuit 804 or the second therapy circuit 808 is integrated into a multi-shock accessory device without monitoring capabilities, and which is solely designed to output electrical shocks upon receiving an input signal from a separate device. For example, the multi-shock accessory device may lack, or be disconnected from, one or more sensors configured to identify one or more physiological parameters of the subject 802. In some cases, the multi-shock accessory device lacks a display, speaker, or other user interface device. According to some cases, the multi-shock accessory device includes one or more capacitors that are charged by a power source from the separate device (e.g., a standalone defibrillator connected to the multi-shock accessory device). In some implementations, the first therapy circuit 804 and the second therapy circuit 808 are integrated into the same device, such as the same monitor-defibrillator.

Optionally, a timing coordinator 820 is configured to cause the first therapy circuit 804 to output the first shock 806 during a first time interval 822 and/or to cause the second therapy circuit 808 to output the second shock 810 during the second time interval 824. For example, the timing coordinator 820 outputs one or more signals (e.g., electrical signals, communication signals, etc.) to the first therapy circuit 804 and/or the second therapy circuit 808. Upon receiving the signal(s) from the timing coordinator 820, the first therapy circuit 804 may discharge the first capacitor 812 during the first time interval 822 and/or the second therapy circuit 808 may discharge the second capacitor 814 during the second time interval 824. The timing coordinator 820 can be implemented in hardware (e.g., a circuit), software (e.g., instructions executed by at least one processor), or a combination thereof. In some cases, the timing coordinator 820 is a standalone device. Examples of standalone timing devices that can serve as the timing coordinator 820 are described in U.S. Pat. No. 10,981,014, which is incorporated by reference herein in its entirety. In some examples, the timing coordinator 820 is integrated into the same device as the first therapy circuit 804 and/or the second therapy circuit 808.

Various timing relationships between the first time interval 822 and the second time interval 824 can be implemented according to various implementations of the present disclosure. In some cases, a delay between the start times (i.e., the leading edges) of the first time interval 822 and the second time interval 824 is in a range of 0 and 250 milliseconds (ms). In some cases, the delay between the start times of the first time interval 822 and the second time interval 824 is in a range of −250 and 0 ms. Although FIG. 8B illustrates the first time interval 822 and the second time interval 824 as having equivalent durations, implementations are not so limited. For example, the first time interval 822 may be longer or shorter than the second time interval 824. Various timing relationships are described in U.S. Pat. No. 10,702,701, which is incorporated by reference herein in its entirety.

In various cases, the timing coordinator 820 is configured to detect the first shock 806 and may cause the second therapy circuit 808 to output the second shock 810 in response. For example, the timing coordinator 820 may detect a signal indicative of the discharge of the first shock 806, and may output a signal that causes the second therapy circuit 808 to discharge the second shock 810. In some examples, the timing coordinator 820 is inductively coupled with the first therapy circuit 804 and/or the first electrodes 816, which enables the timing coordinator 820 to detect the discharge of the first shock 806. Various techniques for detecting the discharge of a first shock in order to cause the application of a second shock in multi-shock therapy are described in U.S. Pat. Nos. 10,625,088 and 10,632,320, which are incorporated by reference herein in their entirety.

### EXAMPLE CLAUSES

The following example clauses provide various implementations of the present disclosure. However, implementations of the present disclosure are not limited to the example clauses provided herein.
1. A system, including: a defibrillator including: a first treatment circuit including a first capacitor configured to discharge a primary electrical shock to first electrodes configured to be disposed on skin of a subject; and a first processor configured to: determine that a multi-shock accessory device is coupled with the defibrillator; and in response to determining that the multi-shock accessory device is coupled with the defibrillator, cause a multi-shock instruction to be provided to the multi-shock accessory device, the multi-shock instruction indicating a time interval between the primary electrical shock and a secondary electrical shock; and the multi-shock accessory device including: a housing configured to removably couple the multi-shock accessory device with the defibrillator; a second treatment circuit including a second capacitor configured to discharge a secondary electrical shock to the subject; and a second processor configured to: receive, from the defibrillator, the multi-shock instruction; and in response to receiving the multi-shock instruction, cause the second treatment circuit to discharge the second capacitor to output the secondary electrical shock to second electrodes configured to be disposed on the skin of the subject, the secondary electrical shock being temporally overlapping with the primary electrical shock.
2. The system of clause 1, wherein the housing of the multi-shock accessory device is configured to dock to the defibrillator via a docking mechanism of the defibrillator.
3. The system of clause 1 or 2, wherein the multi-shock instruction further indicates: an energy level at which to deliver the secondary electrical shock; and the second processor is further configured to cause the second treatment circuit to output the secondary electrical shock at the energy level.
4. A method, including: receiving, by a multi-shock accessory device, a multi-shock instruction from a defibrillator, the multi-shock instruction indicating a time of a primary electrical shock or a secondary electrical shock; and in response to receiving the multi-shock instruction: causing, by the multi-shock accessory device, a treatment circuit of the multi-shock accessory device to output a secondary electrical shock to electrodes configured to be disposed on skin of a subject.
5. The method of clause 4, wherein the multi-shock accessory device includes a mechanical chest compression device, and wherein the mechanical chest compression device is coupled to the electrodes, the method further including: administering, by the mechanical chest compression device, chest compressions to the subject; detecting, via the electrodes, an electrocardiogram (ECG) of the subject; and removing a chest compression artifact from the ECG.
6. The method of clause 4 or 5, wherein the multi-shock accessory device includes a mechanical chest compression device, and wherein the mechanical chest compression device includes a motor to administer chest compressions to the subject, the method further including: causing, by the mechanical chest compression device, the motor to be driven by a power source of the treatment circuit; and administering, by the mechanical chest compression device, the chest compressions to the subject using the motor.
7. The method of any of clauses 4 to 6, further including: receiving, by the multi-shock accessory device, prior to receiving the multi-shock instruction, a charging signal from the defibrillator; and charging, by the multi-shock accessory device, a capacitor of the treatment circuit using the charging signal received from the defibrillator, wherein causing the treatment circuit of the multi-shock accessory device to output the secondary electrical shock to the electrodes includes causing the capacitor to discharge the secondary electrical shock to the electrodes.
8. The method of any of clauses 4 to 7, further including receiving, by the multi-shock accessory device, a user input signal to initiate multi-shock therapy, wherein the multi-shock instruction is received in response to receiving the user input signal.
9. The method of any of clauses 4 to 8, wherein: the multi-shock instruction further indicates a vector, among multiple vectors, for the output of the secondary electrical shock; and the method further includes, determining, by the multi-shock accessory device, that the electrodes are associated with the vector prior to causing: the treatment circuit to output the secondary electrical shock to the electrodes.
10. The method of any of clauses 4 to 9, wherein: the multi-shock instruction further indicates an energy level at which to deliver the secondary electrical shock; and the multi-shock accessory device causes: the treatment circuit to output the secondary electrical shock at the energy level.
11. A multi-shock accessory device for use with a defibrillator, the multi-shock accessory device including: a treatment circuit including a capacitor configured to be charged and subsequently discharged to output electrical shocks; and a processor configured to: receive a multi-shock instruction from the defibrillator, the multi-shock instruction indicating a time interval between a primary electrical shock and a secondary electrical shock; and in response to receiving the multi-shock instruction: cause the capacitor to discharge a secondary electrical shock to electrodes configured to be disposed on skin of a subject.
12. The multi-shock accessory device of clause 11, wherein the multi-shock accessory device includes a mechanical chest compression device configured to administer chest compressions to the subject.
13. The multi-shock accessory device of clause 11 or 12, wherein the multi-shock accessory device includes an automated external defibrillator (AED).
14. The multi-shock accessory device of any of clauses 11 to 13, wherein the multi-shock accessory device includes a therapy delivery portion of a modular defibrillator.
15. The multi-shock accessory device of any of clauses 11 to 14, further including the electrodes.
16. The multi-shock accessory device of any of clauses 11 to 15, further including a housing configured to be removably coupled with the defibrillator.
17. The multi-shock accessory device of clause 16, wherein the housing of the multi-shock accessory device is configured to dock to the defibrillator via a docking mechanism of the defibrillator.
18. The multi-shock accessory device of clause 16 or 17, wherein: the electrodes include three or more electrodes; the housing of the multi-shock accessory device includes: an input port configured to be coupled with a first cable connecting the input port with the defibrillator; and output ports configured to be coupled with second cables connecting the output ports with the three or more electrodes; the primary electrical shock is output to a first pair of the three or more electrodes; and the secondary electrical shock is output to a second pair of the three or more electrodes.
19. The multi-shock accessory device of any of clauses 11 to 18, wherein the multi-shock instruction further indicates: a vector, among multiple vectors, for the output of the secondary electrical shock; or an energy level at which to deliver the secondary electrical shock.
20. The multi-shock accessory device of any of clauses 11 to 19, further including a transceiver configured to receive the multi-shock instruction wirelessly from the defibrillator.
21. The multi-shock accessory device of any of clauses 11 to 20, wherein the multi-shock accessory device lacks a display.
22. A defibrillation pad including the multi-shock accessory device of any of clauses 11 to 21.
23. A system, including: a first defibrillator including: a first treatment circuit including a first capacitor configured to discharge a primary electrical shock to a subject; and a first processor configured to: receive, from a multi-shock accessory device, a first multi-shock instruction; and in response to receiving the first multi-shock instruction, cause the first treatment circuit to discharge the first capacitor to output the primary electrical shock to first electrodes configured to be disposed on skin of the subject; and the multi-shock accessory device including: a housing configured to removably couple the multi-shock accessory device with the first defibrillator; and a second processor configured to: determine that the multi-shock accessory device is coupled with the first defibrillator; in response to determining that the multi-shock accessory device is coupled with the first defibrillator, cause the first multi-shock instruction to be provided to the first defibrillator; and cause a second treatment circuit of the multi-shock accessory device to discharge a second capacitor to output a secondary electrical shock to second electrodes configured to be disposed on the skin of the subject; or in response to determining that the multi-shock accessory device is coupled with the first defibrillator and with a second defibrillator, cause a second multi-shock instruction to be provided to the second defibrillator, the second multi-shock instruction indicating a time interval between the primary electrical shock and the secondary electrical shock and causing the second defibrillator to output the secondary electrical shock to the second electrodes such that the secondary electrical shock temporally overlaps with the primary electrical shock is output by the first defibrillator.
24. The system of clause 23, wherein the housing of the multi-shock accessory device is configured to dock to the first defibrillator via a docking mechanism of the first defibrillator.
25. The system of clause 23 or 24, wherein the first multi-shock instruction further indicates an energy level at which to deliver the primary electrical shock.
26. A method, including: providing, by a multi-shock accessory device, a first multi-shock instruction to a first defibrillator to cause the first defibrillator to output a primary electrical shock to first electrodes disposed on skin of a subject; and causing, by the multi-shock accessory device, a treatment circuit of the multi-shock accessory device to output a secondary electrical shock to second electrodes disposed on the skin of the subject; or providing, by the multi-shock accessory device, a second multi-shock instruction to a second defibrillator, the second multi-shock instruction indicating a time interval between the primary electrical shock and the secondary electrical shock and causing the second defibrillator to output the secondary electrical shock to the second electrodes.
27. The method of clause 26, wherein the multi-shock accessory device includes a mechanical chest compression device, and wherein the mechanical chest compression device is coupled to the second electrodes, the method further including: administering, by the mechanical chest compression device, chest compressions to the subject; detecting, via the second electrodes, an electrocardiogram (ECG) of the subject; and removing a chest compression artifact from the ECG.
28. The method of clause 26 or 27, wherein the multi-shock accessory device includes a mechanical chest compression device, and wherein the mechanical chest compression device includes a motor to administer chest compressions to the subject, the method further including: causing, by the mechanical chest compression device, the motor to be driven by a power source that is also used for defibrillation; and administering, by the mechanical chest compression device, the chest compressions to the subject using the motor.
29. The method of any of clauses 26 to 28, wherein the multi-shock accessory device includes a capacitor configured to discharge the secondary electrical shock, the method further including: receiving, by the multi-shock accessory device, prior to providing the first multi-shock instruction, a charging signal from the first defibrillator; and charging, by the multi-shock accessory device, the capacitor using the charging signal received from the first defibrillator.
30. The method of any of clauses 26 to 29, further including receiving, by the multi-shock accessory device, a user input signal to initiate multi-shock therapy, wherein the first multi-shock instruction is provided in response to receiving the user input signal.
31. The method of any of clauses 26 to 30, wherein: the first multi-shock instruction further indicates a vector, among multiple vectors, for output of the primary electrical shock; and the method further includes, determining, by the multi-shock accessory device, that the first electrodes are associated with the vector prior to providing the first multi-shock instruction to the first defibrillator.
32. The method of any of clauses 26 to 31, wherein the first multi-shock instruction further indicates an energy level at which to deliver the primary electrical shock.
33. A multi-shock accessory device for use with one or more defibrillators, the multi-shock accessory device including a processor configured to: provide a first multi-shock instruction to a first defibrillator to cause the first defibrillator to output a primary electrical shock to first electrodes configured to be disposed on skin of a subject; cause a treatment circuit of the multi-shock accessory device to output a secondary electrical shock to second electrodes configured to be disposed on the skin of the subject; or provide a second multi-shock instruction to a second defibrillator, the second multi-shock instruction indicating a time interval between the primary electrical shock and the secondary electrical shock and causing the second defibrillator to output the secondary electrical shock to the second electrodes.
34. The multi-shock accessory device of clause 33, further including the treatment circuit, wherein the processor is configured to cause the treatment circuit to output the secondary electrical shock to the second electrodes when the primary electrical shock is being output by the first defibrillator.
35. The multi-shock accessory device of clause 33 or 34, further including a capacitor configured to be charged and subsequently discharged, wherein the processor is further configured to cause the capacitor to discharge the secondary electrical shock to the second electrodes.
36. The multi-shock accessory device of any of clauses 33 to 35, wherein the multi-shock accessory device includes a mechanical chest compression device configured to administer chest compressions to the subject.
37. The multi-shock accessory device of any of clauses 33 to 36, wherein the multi-shock accessory device includes an automated external defibrillator (AED) including the treatment circuit, a display, and a speaker, wherein the processor is configured to: deactivate the display and the speaker of the AED; and cause the treatment circuit to output the secondary electrical shock to the second electrodes.
38. The multi-shock accessory device of any of clauses 33 to 37, wherein the multi-shock accessory device includes a therapy delivery portion of a modular defibrillator, the therapy delivery portion including the treatment circuit, wherein the processor is configured to cause the treatment circuit to output the secondary electrical shock to the second electrodes.
39. The multi-shock accessory device of any of clauses 33 to 38, further including the treatment circuit and the second electrodes, wherein the processor is configured to cause the treatment circuit to output the secondary electrical shock to the second electrodes.
40. The multi-shock accessory device of any of clauses 33 to 39, further including a housing configured to be removably coupled with the first defibrillator.
41. The multi-shock accessory device of any of clauses 33 to 40, wherein the first multi-shock instruction further indicates: a vector, among multiple vectors, for delivery of the primary electrical shock; or an energy level at which to deliver the primary electrical shock.
42. The multi-shock accessory device of any of clauses 33 to 41, further including a transceiver configured to send the first multi-shock instruction wirelessly to the first defibrillator.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A system, comprising:
a defibrillator comprising:
a first treatment circuit comprising a first capacitor configured to discharge a primary electrical shock to first electrodes configured to be disposed on skin of a subject; and
a first processor configured to:
determine that a multi-shock accessory device is coupled with the defibrillator; and
in response to determining that the multi-shock accessory device is coupled with the defibrillator, cause a multi-shock instruction to be provided to the multi-shock accessory device, the multi-shock instruction indicating a time interval between the primary electrical shock and a secondary electrical shock; and
the multi-shock accessory device comprising:
a housing configured to removably couple the multi-shock accessory device with the defibrillator;
a second treatment circuit comprising a second capacitor configured to discharge a secondary electrical shock to the subject; and
a second processor configured to:
receive, from the defibrillator, the multi-shock instruction; and
in response to receiving the multi-shock instruction, cause the second treatment circuit to discharge the second capacitor to output the secondary electrical shock to second electrodes configured to be disposed on the skin of the subject, the secondary electrical shock being temporally overlapping with the primary electrical shock.

2. The system of claim 1, wherein the housing of the multi-shock accessory device is configured to dock to the defibrillator via a docking mechanism of the defibrillator.

3. The system of claim 1, wherein the multi-shock instruction further indicates:
an energy level at which to deliver the secondary electrical shock; and
the second processor is further configured to cause the second treatment circuit to output the secondary electrical shock at the energy level.

4. A method, comprising:
receiving, by a multi-shock accessory device, a multi-shock instruction from a defibrillator, the multi-shock instruction indicating a time of a primary electrical shock or a secondary electrical shock; and
in response to receiving the multi-shock instruction:
causing, by the multi-shock accessory device, a treatment circuit of the multi-shock accessory device to output a secondary electrical shock to electrodes configured to be disposed on skin of a subject.

5. The method of claim 4, wherein the multi-shock accessory device comprises a mechanical chest compression device, and wherein the mechanical chest compression device is coupled to the electrodes, the method further comprising:
administering, by the mechanical chest compression device, chest compressions to the subject;
detecting, via the electrodes, an electrocardiogram (ECG) of the subject; and
removing a chest compression artifact from the ECG.

6. The method of claim 4, wherein the multi-shock accessory device comprises a mechanical chest compression device, and wherein the mechanical chest compression device comprises a motor to administer chest compressions to the subject, the method further comprising:
causing, by the mechanical chest compression device, the motor to be driven by a power source of the treatment circuit; and
administering, by the mechanical chest compression device, the chest compressions to the subject using the motor.

7. The method of claim 4, further comprising:
receiving, by the multi-shock accessory device, prior to receiving the multi-shock instruction, a charging signal from the defibrillator; and
charging, by the multi-shock accessory device, a capacitor of the treatment circuit using the charging signal received from the defibrillator,
wherein causing the treatment circuit of the multi-shock accessory device to output the secondary electrical shock to the electrodes comprises causing the capacitor to discharge the secondary electrical shock to the electrodes.

8. The method of claim 4, further comprising receiving, by the multi-shock accessory device, a user input signal to initiate multi-shock therapy, wherein the multi-shock instruction is received in response to receiving the user input signal.

9. The method of claim 4, wherein:
the multi-shock instruction further indicates a vector, among multiple vectors, for the output of the secondary electrical shock; and
the method further comprises, determining, by the multi-shock accessory device, that the electrodes are associated with the vector prior to causing:
the treatment circuit to output the secondary electrical shock to the electrodes.

10. The method of claim 4, wherein:
the multi-shock instruction further indicates an energy level at which to deliver the secondary electrical shock; and
the multi-shock accessory device causes:
the treatment circuit to output the secondary electrical shock at the energy level.

11. A multi-shock accessory device for use with a defibrillator, the multi-shock accessory device comprising:
a treatment circuit comprising a capacitor configured to be charged and subsequently discharged to output electrical shocks; and
a processor configured to:
receive a multi-shock instruction from the defibrillator, the multi-shock instruction indicating a time interval between a primary electrical shock and a secondary electrical shock; and
in response to receiving the multi-shock instruction:
cause the capacitor to discharge a secondary electrical shock to electrodes configured to be disposed on skin of a subject.

12. The multi-shock accessory device of claim 11, wherein the multi-shock accessory device comprises a mechanical chest compression device configured to administer chest compressions to the subject.

13. The multi-shock accessory device of claim 11, wherein the multi-shock accessory device comprises an automated external defibrillator (AED).

14. The multi-shock accessory device of claim 11, wherein the multi-shock accessory device comprises a therapy delivery portion of a modular defibrillator.

15. The multi-shock accessory device of claim 11, further comprising the electrodes.

16. The multi-shock accessory device of claim 11, further comprising a housing configured to be removably coupled with the defibrillator.

17. The multi-shock accessory device of claim 16, wherein the housing of the multi-shock accessory device is configured to dock to the defibrillator via a docking mechanism of the defibrillator.

18. The multi-shock accessory device of claim 16, wherein:
the electrodes comprise three or more electrodes;
the housing of the multi-shock accessory device comprises:
an input port configured to be coupled with a first cable connecting the input port with the defibrillator; and
output ports configured to be coupled with second cables connecting the output ports with the three or more electrodes;
the primary electrical shock is output to a first pair of the three or more electrodes; and
the secondary electrical shock is output to a second pair of the three or more electrodes.

19. The multi-shock accessory device of claim 11, wherein the multi-shock instruction further indicates:
a vector, among multiple vectors, for the output of the secondary electrical shock; or
an energy level at which to deliver the secondary electrical shock.

20. The multi-shock accessory device of claim 11, further comprising a transceiver configured to receive the multi-shock instruction wirelessly from the defibrillator.
